(19) United States
(12) Patent Application Publication
(10) Pub. No.: US 2015/0065994 A1
(43) Pub. Date: Mar. 5, 2015

(54) POSTERIOR BREAST MASSAGE UNIT

(71) Applicant: Positive Care Ltd., Nazareth IIit (IL)

(72) Inventors: Ofer FRIDMAN, Lotem (IL); Zahia Kasseml, Kfar Nahif (IL); Dov Barkay, Ramat-Yishai (IL)

(73) Assignee: Positive Care Ltd., Nazareth IIit (IL)

(21) Appl. No.: 14/260,433

(22) Filed: Apr. 24, 2014

Related U.S. Application Data
(60) Provisional application No. 61/983,511, filed on Apr. 24, 2014.
(60) Provisional application No. 61/871,434, filed on Aug. 29, 2013.

(51) Int. Cl. A61M 1/06

(52) U.S. Cl. CPC A61M 1/068
USPC 604/514; 604/74; 604/66

(57) ABSTRACT

A method of milk extraction system may include massaging a posterior of a breast. Massaging and collecting milk may optionally be performed simultaneously. A posterior massager may also be used and/or mounted separately from a collection unit. In some embodiments the massager may be used to improve milk collection and/or alleviate breast ailments for example engorgement, plugged ducts, and/or mastitis. A collection flange may include an air inlet manifold, to mix air and milk. Optionally as air is drawn along a tube by a vacuum, milk may be entrained in the airstream and brought towards a collection vessel. The system may include automated tracking of milk production and/or consumption.

### RELATED APPLICATIONS

This application claims the benefit of priority under 35 USC §119(e) of U.S. Provisional Patent Application Nos. 61/871,434 filed Aug. 29, 2013 and 61/983,511 filed Apr. 24, 2014. The contents of the above applications are incorporated herein by reference in their entirety.

### FIELD AND BACKGROUND OF THE INVENTION

The present invention, in some embodiments thereof, relates to a breast massage and/or milk extraction unit and, more particularly, but not exclusively, to a faster and healthier method of expressing milk.

U.S. Patent Application Publication No. 2005/0234370 discloses “a breast pump that supports a mother's and infant's breastfeeding needs and facilitates the lifestyle of the mother's choice. The breast milk expression system disclosed allows breastfeeding mothers to obtain milk comfortably, hands-free, conveniently and with discretion. The breast milk expression system disclosed includes at least two contact points which mimic hand-expression as well as infant suckling and effectively stimulate the secretion of oxytocin and prolactin to provide a healthy, safe and efficient breastfeeding session. The system disclosed has a gentle rhythmic massage means and is located a distance from the base of the nipple and massages in all quadrants of the breast. The rhythmic forward pressure is provided by a plurality of opposing pairs of expression bellows that move the milk from the lactiferous sinuses through the nipple pores, bio-mimicking the techniques of hand expression”.

U.S. Patent Application Publication No. 20090/156993 discloses a breast milk collection and storage device that contours to the natural form of a female human breast and consists of an outer layer, an inner layer and a sterile collection bag. Designed to fit under any standard brassiere, the device provides an opening to be comfortably worn around the nipple. The opening directs milk to the sterile collection bag and further provides a sealing means for coupling to a cap or other closure device when not being worn.

U.S. Pat. No. 7,785,305 discloses a portable, hands-free and user friendly breast pump for facilitating breast-feeding. The pump incorporates a dome-shaped housing having a servomotor mechanism; a hat-shaped flange having a chamber portion, a brim portion and an outlet, the chamber portion being formed so as to be placed over a nipple on a breast so as to define a chamber between the flange and the breast, and the brim portion being formed to surround the nipple and thereby form a suction seal therebetween; and a one-way venting element formed to operatively communicate with the chamber so as to vent pumped milk from the chamber via the outlet. The housing, the flange and the venting element are connected into an integral device held against the breast and underneath clothing so as to keep the integral device hidden. Breast milk is collected via a plastic collection bag that connects directly with the pump underneath clothing such that the milk is isolated from any contaminants in the air and may be easily stored immediately after being collected.

European Patent Application Publication No. 2502640 discloses “a breast pump comprising a funnel and at least two cone-shaped breast pump inserts interchangeably mountable in said funnel” . . . . “The breast pump inserts each have a central aperture therein through which a nipple protrudes when a breast is received in the funnel. The diameter of the aperture in each breast pump insert is different. A breast pump funnel or insert having a textured finish on a breast interface surface is also disclosed”.

Maya Bolman et al. in “Recapturing the Art of Therapeutic Breast Massage during Breastfeeding” Journal of Human Lactation vol. 29 issue 3 pages 328-331, 2013, DOI: 10.1177/0890334413475527, teach that “Russian clinicians routinely combine hand expression with breast massage for the treatment of milk stasis, engorgement, and plugged ducts”.

Morton et al. “Combining hand techniques with electric pumping increases milk production in mothers of preterm infants” Journal of Perinatology vol. 29, pages 757-764, 2009 teach that “pump suction alone often fails to remove a significant fraction of milk as more can be expressed using manual techniques. Before mature milk ‘comes in’, hand expression may more effectively remove colostrum. Once volume increases, a combination of pump suction, breast compression and, if needed, hand expression collectively referred to as ‘hands-on pumping’ (HOP) may remove a greater fraction of milk than pump suction alone”. Morton et al. further observed that “For decades, efforts to improve production have focused primarily on pumping schedules and pump design (that is, electrical vs. manual, single vs. double, and vacuum patterns) . . . maternal demographics, perinatal issues . . . pumping initiation (time between birth and first expression), frequency and duration, and the longest interval between two pumping sessions (potential sleeping time). However, few studies have evaluated the use of manual techniques to increase milk yield and/or milk quality”.

Additional background art includes: N. P. Alekseeva, V. I. Ilyina, V. K. Yaroslayski, S. N. Gaidukov, T. K. Tikhonova, Y. A. Specivcev, E. V. Omelyanjuk, N. N. Tkachenko; Compression stimuli increase the efficacy of breast pump function; European Journal of Obstetrics & Gynecology and Reproductive Biology, volume 77 (1998), pages 131-139.

### SUMMARY OF THE INVENTION

According to an aspect of some embodiments of the present invention there is provided a method of extracting milk from a breast of a woman including: securing a massager to the breast; massaging a posterior region of the breast with the massager, the massaging continuing least intermittently over a massage time period; and extracting milk from the breast during the massage time period.

According to some embodiments of the invention, the method further includes positioning a flange over a nipple of the breast subsequent to the securing and wherein the extracting is through the flange.

According to some embodiments of the invention, the positioning includes inserting the breast into the flange through an opening of the flange and wherein the massaging the posterior region includes a region posterior to the opening and at least 2 cm distant from the opening.

According to some embodiments of the invention, the extracting includes applying an oscillating vacuum reaching a maximum magnitude of less than 100 mmHg over a first extracting period of at least four minutes.

According to some embodiments of the invention, the method further includes applying an oscillating vacuum over a first extracting period of at least four minutes and wherein during a maximum vacuum portion of the first extracting period a nipple of the breast is drawn into a nipple channel of the flange to a distance of less than 2 cm.

According to some embodiments of the invention, the posterior region includes a location less than 3 cm from a chest wall of the woman.

According to some embodiments of the invention, the posterior region includes three points defining a plane posterior to 50% of the volume of the breast.

According to some embodiments of the invention, the method further includes detecting a volume of the breast and adjusting a parameter of the massaging in response to the detecting.

According to some embodiments of the invention, the method further includes detecting a volume of the breast and adjusting a parameter of the extracting in response to the detecting.

According to an aspect of some embodiments of the present invention there is provided a system for extracting milk from a woman including: a breast massager; a retainer securing the breast massager to a posterior region of a breast; and an opening in the breast massager and retainer allowing access to a nipple of the breast.

According to some embodiments of the invention, the opening is less than 6 cm wide.

According to some embodiments of the invention, the system further includes a flange fitting through the opening and over the nipple, and a collection unit collecting the milk from the flange into a collection vessel.

According to some embodiments of the invention, the system further includes a source of a vacuum and wherein the flange includes a nipple channel and wherein the source is connected to the flange to draw the nipple into the nipple channel and wherein a length of the nipple channel is less than 2 cm.

According to some embodiments of the invention, the retainer secures the massager to the posterior region of the breast, posterior to the flange and at a distance of at least 2 cm from the flange.

According to some embodiments of the invention, the system further includes a controller programmed to direct massaging of the posterior region of the breast by the breast massager and simultaneously to direct the collecting the milk by the collection unit.

According to some embodiments of the invention, the posterior region includes a location less than 3 cm from a chest wall of the woman.

According to some embodiments of the invention, the posterior region includes three points defining a plane posterior to 50% of the volume of the breast.

According to some embodiments of the invention, the massager includes one or more inflatable massage element.

According to some embodiments of the invention, the retainer includes a channel and wherein upon inflation the massage elements fill the channel.

According to some embodiments of the invention, the system further includes a breast volume sensor and a controller is configured to detect a change in volume of the breast from an output of the breast volume sensor.

According to some embodiments of the invention, the system further includes a flange fitting through the opening and over the nipple, and a collection unit collecting the milk from the flange into a collection vessel wherein the controller is configured to change a parameter of the collecting in response to the change in volume.

According to some embodiments of the invention, the changing a parameter includes stopping the collecting.

According to some embodiments of the invention, the controller is configured to change a parameter of the massaging in response to the change in volume.

According to some embodiments of the invention, the massager includes an inflatable element and the sensor includes a pressure sensor for sensing a pressure of inflation of the inflatable element.

According to some embodiments of the invention, the system further includes an alerter controlled by the controller for notifying a user when a rate the change of the volume is below a threshold.

According to an aspect of some embodiments of the present invention there is provided a method of extracting milk from a breast of a woman comprising: securing a massager to the breast; massaging a posterior region of the breast with the massager, the massaging continuing least intermittently over a massage time period; and extracting milk from the breast during the massage time period.

According to some embodiments of the invention, the method further includes: positioning a flange over a nipple of the breast subsequent to the securing and wherein the extracting is through the flange.

According to some embodiments of the invention, the positioning includes inserting the breast into the flange through an opening of the flange and wherein the massaging the posterior region includes a region posterior to the opening and at least 2 cm distant from the opening.

According to some embodiments of the invention, the extracting includes applying an oscillating vacuum reaching a maximum magnitude of less than 100 mmHg over a first extracting period of at least four minutes.

According to some embodiments of the invention, the extracting includes applying an oscillating vacuum over a first extracting period of at least four minutes and wherein during a maximum vacuum portion of the first extracting period a nipple of the breast is drawn into a nipple channel of the flange to a distance of less than 2 cm.

According to some embodiments of the invention, the posterior region includes a location less than 3 cm from a chest wall of the woman.

According to some embodiments of the invention, the posterior region includes three points defining a plane posterior to 50% of the volume of the breast.

According to some embodiments of the invention, the method further includes:

detecting a volume of the breast and adjusting a parameter of the massaging in response to the detecting.

According to some embodiments of the invention, the method further includes:

detecting a volume of the breast and adjusting a parameter of the extracting in response to the detecting.

According to some embodiments of the invention, the massaging includes compressing the posterior region to facilitate the extracting.

According to some embodiments of the invention, the securing includes encircling the posterior region.

According to an aspect of some embodiments of the present invention there is provided a system for extracting milk comprising: a breast massager; a retainer securing the breast massager to a posterior region of a breast; and an opening in the breast massager and retainer allowing access to a nipple of the breast.

According to some embodiments of the invention, the opening is less than 6 cm wide.

According to some embodiments of the invention, the system further includes a flange fitting through the opening and over the nipple, and a collection unit collecting the milk from the flange into a collection vessel.

According to some embodiments of the invention, the system further includes a source of a vacuum and wherein the flange includes a nipple channel and wherein the source is connected to the flange to draw the nipple into the nipple channel and wherein a length of the nipple channel is less than 2 cm.

According to some embodiments of the invention, the retainer secures the massager to the posterior region of the breast, posterior to the flange and at a distance of at least 2 cm from the flange.

According to some embodiments of the invention, the system further includes a controller programmed to direct massaging of the posterior region of the breast by the breast massager and simultaneously to direct the collecting the milk by the collection unit.

According to some embodiments of the invention, the posterior region includes a location less than 3 cm from a chest wall of the woman.

According to some embodiments of the invention, the posterior region includes three points defining a plane posterior to 50% of the volume of the breast.

According to some embodiments of the invention, the massager includes one or more inflatable massage element.

According to some embodiments of the invention, the retainer includes a channel and wherein upon inflation the massage elements fill the channel.

According to some embodiments of the invention, the system further includes a breast volume sensor and a controller is configured to detect a change in volume of the breast from an output of the breast volume sensor.

According to some embodiments of the invention, the system further includes a flange fitting through the opening and over the nipple, and a collection unit collecting the milk from the flange into a collection vessel wherein the controller is configured to change a parameter of the collecting in response to the change in volume.

According to some embodiments of the invention, the changing a parameter includes stopping the collecting.

According to some embodiments of the invention, the controller is configured to change a parameter of the massaging in response to the change in volume.

According to some embodiments of the invention, the massager includes an inflatable element and the sensor includes a pressure sensor for sensing a pressure of inflation of the inflatable element.

According to some embodiments of the invention, the system further includes an alerter controlled by the controller for notifying a user when a rate the change of the volume is below a threshold.

According to some embodiments of the invention, the collection vessel is in a collapsed configuration before collecting and the collection vessel is in an expanded configuration after collecting.

According to some embodiments of the invention, the retainer includes a brassiere.

According to an aspect of some embodiments of the present invention there is provided a method of extracting milk from a breast including: securing a massager to the breast; massaging a posterior region of the breast with the massager; and collecting milk from the breast simultaneous to the massaging.

According to some embodiments of the invention, the method may further include exposing a nipple and areola of the breast subsequent to the securing and wherein the collecting is by a baby suckling to the nipple.

According to some embodiments of the invention, the method may further include exposing a nipple of the breast subsequent to the securing; and positioning a flange over the nipple subsequent to the exposing and wherein the collecting is through the flange.

According to some embodiments of the invention, the method may further include sensing rate of the collecting.

According to some embodiments of the invention, the sensing includes at least one measuring at least one parameter selected from a tension on a collection vessel, a volume stored in a collection vessel, mass stored in a collection vessel, a volume of the breast and a flow rate in a collection pathway.

According to some embodiments of the invention a collection pathway connects the breast to a collection vessel. The method may further include drawing air through the collection pathway with a net flow towards the collection vessel.

According to some embodiments of the invention, the method may further include entraining the milk into the net flow.

According to some embodiments of the invention, the method may further include securing said massager by means of a brassiere.

According to some embodiments of the invention, securing includes encircling a portion of the breast with the massager.

According to some embodiments of the invention, the encircling includes encircling the posterior region.

According to some embodiments of the invention, the collecting is to a collection vessel and the collection vessel is in a collapsed configuration before the collecting and the collection vessel is in an expanded configuration after the collecting.

According to some embodiments of the invention, the method may further include expanding the collection vessel into the expanded configuration with a frame.

According to some embodiments of the invention, the method may further include switching the massaging from a first manner to a different manner.

According to some embodiments of the invention, the switching is between a plurality of massage manners in turn according to a schedule.

According to some embodiments of the invention, the massaging is adapted to alleviate at least one breast ailment selected from the group consisting of engorgement, plugged ducts, and mastitis.

According to some embodiments of the invention, the massaging includes at least one action selected from the group consisting of quick application and quick release of pressure, slow application and release of pressure, an extended massage regime, applying and releasing pressure to a large area of the breast, vibrating the breast, focusing the massaging on a hard spot on the breast, focusing the massaging on a painful spot on the breast, applying pressure from an anterior region of the breast towards the posterior region and applying the massaging when the breast is affected by an ailment.

According to some embodiments of the invention, the massaging includes compressing the posterior region to facilitate the collecting.

According to some embodiments of the invention, the method may further include securing a massager to a breast and massaging the breast with the massager in a manner adapted to alleviate at least one ailment selected from the group consisting of engorgement, plugged ducts, and mastitis, and wherein the massaging includes at least one action selected from the group consisting of quick application and quick release of pressure, slow application and release of pressure, an extended massage regime, applying and releasing pressure to a large area of the breast, vibrating the breast, focusing the massaging on a hard spot on the breast, focusing the massaging on a painful spot on the breast, applying pressure from an anterior region of the breast towards a posterior region and applying the massaging when the breast is affected by an ailment.

According to an aspect of some embodiments of the present invention there is provided an apparatus for collecting milk including a flange fitting over a nipple of the breast; a collection vessel; a collection pathway leading from the flange to the collection vessel; an air inlet; an air outlet; and a pump producing airflow from the air inlet to the air outlet; the airflow including stream having a net movement toward the collection vessel in a at least a portion of the collection pathway.

According to some embodiments of the invention, the apparatus may further include a manifold entraining the milk into the stream.

According to some embodiments of the invention, the air inlet is located on the flange.

According to some embodiments of the invention, the air inlet is located between the flange and the collection vessel.

According to some embodiments of the invention, the collection vessel is in a collapsed configuration before the collecting and the collection vessel is in an expanded configuration after the collecting.

According to some embodiments of the invention, the collection vessel includes a frame holding the collection vessel in the extended configuration.

According to some embodiments of the invention, the apparatus may further include a sensor for measuring a rate of the collecting.

According to some embodiments of the invention, the sensor measures at least one parameter selected from the group consisting of a tension on the collection vessel, a volume stored in the collection vessel, mass stored in a collection vessel, a volume of the breast and a flow rate in a collection pathway.

According to some embodiments of the invention, the apparatus may further include a massager for a posterior of the breast.

According to some embodiments of the invention, the apparatus may further include a controller coordinating simultaneously functioning of the massager and the vacuum.

According to an aspect of some embodiments of the present invention there is provided a system for extracting milk including: a breast massager encircling a posterior region of a breast; and an opening in the breast massager exposing a nipple and an areola of the breast.

According to some embodiments of the invention, the opening is sufficient for a baby to nurse from the nipple.

According to some embodiments of the invention, the system may further include a flange fitting through the opening and over the nipple, and a collection unit collecting the milk from the flange into a collection vessel.

According to some embodiments of the invention, the breast massager surrounds at least 70% of a circumference of the breast.

According to some embodiments of the invention, the system may further include a brassiere and wherein the brassiere secures the breast massager to the breast.

According to some embodiments of the invention, the system may further include a massager secured to a posterior region of a breast; a collection unit collecting the milk from the breast into a collection vessel; and a controller programmed to direct massaging of the posterior region of the breast by the breast massager and simultaneously to direct the collecting the milk by the collection unit.

According to some embodiments of the invention, the system may further include a flange; a brassiere securing the massager to a posterior region of the breast and holding the flange over a nipple of the breast; and a connector reversibly connecting the flange to the collection unit.

According to some embodiments of the invention, the system may further include at least one drive simultaneously powering the massager and the collection unit.

According to some embodiments of the invention, the system may further include a collection pathway leading from the breast to the collection vessel; an air inlet; an air outlet; and a pump producing airflow from the air inlet to the air outlet; the airflow including stream having a net movement toward the collection vessel in a at least a portion of the collection pathway.

According to some embodiments of the invention, the system may further include a manifold entraining the milk into the stream.

According to some embodiments of the invention, the air inlet is located on the flange.

According to some embodiments of the invention, the air inlet is located between the flange and the collection vessel.

According to some embodiments of the invention, the air inlet is controlled to prevent foaming or whipping of the milk.

According to some embodiments of the invention, the collection vessel is in a collapsed configuration before the collecting and the collection vessel is in an expanded configuration after the collecting.

According to some embodiments of the invention, the collection vessel includes a frame holding the collection vessel in the extended configuration.

According to some embodiments of the invention, the collection unit further includes a sensor for measuring a rate of the collecting.

According to some embodiments of the invention, the sensor measures at least one parameter selected from the group consisting of a tension on the collection vessel, a volume stored in the collection vessel, mass stored in the collection vessel, a volume of the breast and a flow rate in a collection pathway.

According to an aspect of some embodiments of the present invention there is provided a milk extraction system including: a sensor measuring a volume extracted; a donor identifier; a milk collection vessel; a label marking the milk collection vessel; and a controller recording data including the volume, and a user identification associating the data with the label.

According to some embodiments of the invention, the controller further records the date and/or time.

According to some embodiments of the invention, the sensor includes a breast volume gauge.

According to some embodiments of the invention, the milk collection vessel includes a collection bag.

According to some embodiments of the invention, the collection bag includes a frame.

According to some embodiments of the invention, the frame includes at least one member selected from the group consisting of an inflatable member, a flexible structural member, and a collapsible member.

According to some embodiments of the invention, the system may further include an alerter controlled by the controller for notifying a user when a rate change of the volume is below a threshold.

According to some embodiments of the invention, the system may further include a breast posterior massager.

According to some embodiments of the invention, the system may further include a communication device transferring data.

According to some embodiments of the invention, the data may be transferred to at least one receiver selected from the group consisting of a hospital computer, a pediatrician, an electronic label and/or a printed label, a smartphone, and/or a computer.

According to some embodiments of the invention, the system may further include a picture of a baby displayed on a user display.

According to some embodiments of the invention, the system may further include a video of a baby displayed on a user display.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart illustrating a method of milk extraction in accordance with an embodiment of the present invention;

FIG. 2A is a high level block diagram of components a milk extraction and tracking system according to an embodiment of the present invention;

FIG. 2B is a high level block diagram of a coordinated milk extraction and tracking system according to an embodiment of the present invention;

FIG. 3 is a state diagram of a milk extraction system in accordance with an embodiment of the present invention;

FIG. 4A is a schematic drawing of a milk extraction system in accordance with an embodiment of the present invention;

FIG. 4B is a schematic drawing of a breast squeezing ring of a milk extraction system in accordance with an embodiment of the present invention;

FIG. 4C is a schematic drawing of a nipple massager of a milk extraction system in accordance with an embodiment of the present invention;

FIG. 4D is a milk collection unit with a manifold collection flange in accordance with an embodiment of the present invention;

FIG. 4E is a drawing illustrating use of a milk collecting system with a manifold in accordance with an embodiment of the present invention;

FIG. 5A is a milk collection unit with a standard collection flange in accordance with an embodiment of the present invention;

FIG. 5B is a drawing illustrating use of a milk collecting system without a manifold in accordance with an embodiment of the present invention;

FIG. 6 is a donor unit of a milk collecting system in accordance with an embodiment of the present invention;

FIG. 7A is a drawing of an expandable collection bag for a milk extraction system in accordance with an embodiment of the present invention;

FIG. 7B is a drawing of an expandable collection vessel for a milk extraction system in a collapsed configuration in accordance with an embodiment of the present invention;

FIG. 7C is a drawing of an expandable collection vessel for a milk extraction system in an expanded configuration in accordance with an embodiment of the present invention;

FIGS. 8A-C are drawings of flanges of a milk extraction system in accordance with some embodiments of the present invention;

FIG. 9 is a drawing of an alternative embodiment of leafed breast massager in accordance with an embodiment of the present invention;

FIG. 10A is an exploded view of an alternative embodiment of a breast massager in accordance with an embodiment of the present invention;

FIG. 10B is cross sectional view of an alternative embodiment of a breast massager in accordance with an embodiment of the present invention;

FIG. 11A is a cross section view of an alternative embodiment of a roller breast massager in accordance with an embodiment of the present invention;

FIG. 11B is a front view of an alternative embodiment of a roller breast massager in accordance with an embodiment of the present invention;

FIG. 12 is a schematic depiction of breast anatomy and an exemplary pneumatic massage element in accordance with an embodiment of the present invention;

FIG. 13 is a schematic depiction of breast anatomy and a retainer and pneumatic massage elements in accordance with an embodiment of the present invention;

FIGS. 14A-B are schematic depictions of an exemplary retainer in accordance with an embodiment of the present invention;

FIGS. 15A-B are schematic depictions of tubing connectors accordance with an embodiment of the present invention;

FIGS. 16, 17 and 18 are schematic depictions of regulators in accordance with some embodiments of the present invention;

FIG. 19 is a photographic illustration of an exemplary experimental system for massaging a breast and extracting milk in accordance with some embodiments of the present invention; and

FIG. 20 is a graph showing a typical two period extraction session in accordance with some embodiments of the present invention.

### DESCRIPTION OF SPECIFIC EMBODIMENTS OF THE INVENTION

The present invention, in some embodiments thereof, relates to a breast massage and/or milk extraction unit and, more particularly, but not exclusively, to a faster and healthier method of expressing milk.

### Overview

1 Retainer and/or Massager for Posterior Region of Breast

Some aspects of the present invention relate to a massager of the posterior part of a human breast.

In some embodiments, a massager may include a massaging element located behind a milk collection flange and/or massaging area of the breast behind the milk collection flange. In some embodiments the breast may be inserted into an opening of the flange. For example the opening of the flange may have a width and/or diameter of less than 6 cm and/or ranging between 6 and 7 cm. For example, the massage area may include a location may be between 0 cm and 1 cm beyond the end of the flange. Alternatively or additionally, the massage area may include a location be between 1 cm to 2 cm beyond the end of the flange. Alternatively or additionally, the massage area may include a location be between 2 cm to 4 cm beyond the end of the flange. Alternatively or additionally, the massage area may include a location be between 4 cm to 6 cm beyond the end of the flange.

In some embodiments, a massager may massage and/or compress the posterior portion of the breast (for example the posterior portion of the breast may include a location behind the nipple more than eight cm away from the nipple along the surface of the breast and between five to eight cm and/or between three to five cm away from the nipple along the surface of the breast. Alternatively or additionally, the posterior region may include a location behind the areola more than five cm away from the edge of the areola along the surface of the breast and between two to five away from the areola along the surface of the breast. Alternatively or additionally, the posterior region may include a location on the breast within two cm of where the breast meets the chest wall and/or between two and five cm and/or more than five cm from where the breast meets the chest wall. For example, the posterior region may include a location on the breast that is more than three quarters of the distance from the areola to the chest wall along the surface of the breast and/or between one half and three quarters of the distance from the areola to the chest wall along the surface of the breast. Alternatively or additionally, the posterior region may include a location posterior to 80% of the bra cup and/or posterior to between 70% to 80% of the bra cup and or posterior to between 50% and 70% of the bra cup. The distances along the breast may be measured along the top of the breast and/or along the outside of the breast and/or along the bottom of the breast.

In some embodiments, the massager may have an opening. The opening may optionally expose a nipple and/or an areola. The opening may optionally be sufficient for a baby to nurse through the opening. The opening may optionally be sufficient for positioning a milk collection flange through the opening over the nipple. For example the opening may be of a stretching type (for example a hole surrounded by a rubber and/or elastomer band) and/or a slit type. The opening may for example range between 3 and 6 cm long when stretched. For example the flange may pertain to a separate milk collection unit. Alternatively or additionally, the collection unit and/or extraction unit and/or massager may be integrated into a single device.

In some embodiments massage elements may be held onto the breast by a retainer. For example, the retainer may have a form similar to a brassiere. Optionally, the massage elements may be inflatable. For example an inflatable massage element may inflate to compress the posterior region of the breast. For example the retainer may include a channel. For example upon inflation the inflatable message element may deform and fill the channel and/or take the shape of the channel for example like an inner tube in a tire. In some embodiments of this patent the channel or channels may be made from elastic material. Alternatively or additionally, the channel or channels may be made entirely or partially from inelastic materials. For example, the inner part of the channel may be made from an elastic material and/or the outer part from a non-elastic material. In some embodiments the bra cup may be part of a nursing brassiere. Optionally the breast massager may in some embodiments increase quantity and/or quality of milk production. Optionally the breast massager may in some embodiments help to resolve breastfeeding complications including engorgement, plugged ducts, and/or mastitis. Messaging and/or compression of the posterior region of the breast may sometimes facilitate milk let down and/or speed up milk expression. Massaging and/or compression may have other positive effects such as promoting labor, speeding the labor process, increasing oxytocin production, increasing serum prolactin levels and/or increasing breast size. Along with massaging a posterior portion of breast a massager may optionally massage other areas of the breast. Optionally massaging may be performed during milk collecting and/or before milk collecting and/or after milk collecting and/or at times of breast pain. In some embodiments, massaging may be from the posterior of the breast toward the anterior (for example towards the areola), for example to encourage milk expression. Alternatively or additionally, massaging may be from the anterior of the breast towards the posterior, for example to encourage flow in lymph nodes for example to reduce swelling, engorgement, plugged ducts and/or mastitis.

### 2 Massaging the Posterior of the Breast

Some aspects of the present invention relate to a method of massaging a posterior part of a human breast with a massaging apparatus. Massaging may be applied during and/or previous to milk extraction. The massager may be used for example during milk expression (for example with a breast pump) and/or during breast feeding. Alternatively or additionally, the massager may be used before feeding and/or before milk expression and/or after feeding and/or after expression and/or when not feeding or expressing milk. In some embodiments massaging a posterior region may speed up the milk extraction process, help alleviate nursing ailments (for example engorgement, plugged ducts, and/or mastitis), and/or release hind milk (possibly having advantages such as higher fat content over other milk).

In some embodiments massaging may optionally include some or all of the following techniques for example to facilitate milk extraction and/or treat and/or prevent engorgement, plugged ducts, and/or mastitis: quick application and release of relatively low pressure for example by inflation and deflation of a pneumatic massager (for example the length of each portion of a cycle of pressure application and/or holding and/or release may range between every 0.5 to 2 sec and/or 2 to 5 sec.); slow application and release of relatively low pressure for example by inflation and deflation of a pneumatic massager (for example cycles of pressure and/or release ranging between every 5 to 30 sec); starting with low pressure and slowly increasing the pressure over a period ranging for example between 1 to 5 min; an extended massage regime (for example continuing over a time period ranging between 15 to 45 min; applying pressure first to an anterior portion of the breast and continuing towards the posterior; applying and/or releasing pressure to a large area of the breast (for example inflating most and/or all of the elements of a pneumatic massager simultaneously), vibrating the massager by very quick small changes in pressure and/or pressure waves (for example ultrasonic and/or subsonic vibrations); focusing massage, pressure and/or vibrations on hard spots and/or painful spots in the breast.

In some embodiments a pneumatic massage element may be inflated in each inflation cycle until a prescribed pressure is reached. For example the prescribed pressure may range between 40 to 90 mmHg and/or 90 to 150 mm Hg and/or 150 to 200 mmHg. In some embodiments the volume of inflation may be measured. For example the time to inflate may be used as a measure for volume of inflation (for example assuming consistent rate inflation). Optionally, changes of inflation volume over time will be recorded. For example a controller may track changes in inflation volume. For example the controller may manage massaging and/or extraction based on inflation volume. For example, extraction may start at a low suction (for example ranging between 60 to 120 mmHg). As milk is extracted, the breast may become softer and/or the inflation volume and/or the inflation time may increase. The low extraction suction may be used until the inflation volume ceases to increase over time and/or the rate of increase falls below a threshold (For example a fixed threshold may be used, for example no measured increase and/or an increase of less than 0.1% in two minutes and/or an increase of less than 0.2% in one minute. Alternatively or additionally a proportional threshold may be used, for example less than 20% and/or between 20% to 40% and/or between 40% to 75% of the rate of increase in inflation volume over time at the beginning of the extraction). When the rate of increase of inflation volume over time falls below the threshold, the inflation massage inflation pressure may be increased (for example massaging pressure may be increased from between 60 to 90 mmHg to between 90 to 150 mmHg and/or to greater than 150 mmHg alternatively or additionally pressure may be increased from between 90 to 150 mmHg to between 150 to 250 mmHg) and/or the extraction vacuum may be increased (for example the maximum vacuum level in the cycle may be increased from between 60 to 120 mmHg to between 120 to 220 mmHg and/or to between 220 to 300 mmHg) and/or the extraction session may be stopped. When the inflation volume ceases to increase at a rate above a threshold under the higher pressure and/or vacuum extraction regime, the extraction session may optionally be ended.

In some embodiments, application of a suction cycle may depend on time. For example, suction may be applied at a low vacuum cycle (for example a cycle having a maximum vacuum of less than 100 mmHg) for between 1 to 5 minutes and/or between 5 to 10 minutes and/or between 10 and 15 minutes. Alternatively or additionally, (for example in place of or after the period of low vacuum cycle), suction may be applied at a medium vacuum cycle (for example a cycle having a maximum vacuum ranging between 100 to 180 mmHg) for between 1 to 5 minutes and/or between 5 to 10 minutes and/or between 10 to 15 minutes. For example, in place of and/or along with other suction periods) suction may be applied at a high vacuum cycle (for example a cycle having a maximum vacuum of ranging between 180 to 300 mmHg and/or greater than 180 mmHg) for between 0 to 5 minutes and/or between 5 to 10 minutes.

In some embodiments, the controller may account for time of the extraction session. For example the pressure may start at a low level and may not be increased until a minimum time has elapsed. For example the minimum time for the low pressure cycle may range between one to three minutes. Alternatively or additionally the session may not be stopped until a specified time has passed for example ranging between 8 to 12 minutes and/or between 12 to 20 minutes. Alternatively or additionally the time of extraction at high pressure may be limited based on the time under low pressure and/or low vacuum extraction. For example after low pressure and/or low vacuum extraction for a given time, higher pressure and/or higher vacuum extraction may be applied for a time ranging between 10% to 40% of the time that the low pressure and/or low vacuum extraction was used.

In some embodiments suction may be applied to the breast to extract milk in synchronization with the massaging. For example suction may be applied simultaneous to massaging and/or immediately after massaging. Suction may be applied cyclically in synchronization with message cycles. For example, a massager may cycle between putting pressure on a breast and releasing pressure and/or a collection unit may cycle between suction and neutral pressure. Optionally the suction and pressure on the breast may be applied at the same time. The suction cycle and/or massage cycle may have the same rate. For example the cycling rate may range between one every 0.5 to 2 sec and/or 2 to 5 sec.

In some embodiments a massage unit may apply pressure and/or vibration to a breast. The pressure may range, for example between 10 and 350 mm HG. Low pressure may range for example from between 10 and 100 mm HG. For example, a pneumatic and/or hydraulic element may apply pressure to a breast. The element may have the form of a tube or a leaf. The outer wall of the element may be thicker and/or shorter than the inner wall, for example so that the element will tend to squeeze inward. For example, a cross section of a pneumatic and/or hydraulic tube may optionally include outer wall of length ranging between for example 10 to 40 mm and an inner wall of length ranging between for example 10 to 100 mm. For example the element may include a tube have a resting diameter ranging between 10 to 50 mm and a stretched diameter ranging between 15 to 100 mm. A massage element may simulate a baby rooting and/or suckling (for example a portion of a massage element under the breast may be larger than at other points around the breast). Optionally an under nipple elements may periodically push up on the nipple like a baby rooting and/or suckling. Alternatively or additionally, in some embodiments, some of the massaging elements may include bumps. For example the bumps may simulate the feel of baby fingers grasping a breast.

The present invention in some embodiments thereof is related to a breast massager that may be useful for, but is not limited to healing the breast after surgery, strengthening muscles of the breast, and/or shaping the breast. For example, a massager may also improve healing of the breast, for example after surgery and/or the massager may strengthen the muscles in the breast and/or shape the breast.

### 3 Discreet Milk Extraction for Working Women

An aspect of the present invention relates to a breast massager that is convenient and/or inconspicuous when not in use and/or discreet in use. In some embodiments a breast massager will be associated with a breast pump, for example including a vacuum pump. For example, the massager may massage the breast while a pump applies suction for milk extraction. The massager may optionally be mounted to a breast and/or operated separately from the breast pump. For example, a brassiere may be supplied with a massage unit and/or a connection to allow mounting and/or removal of milk extraction equipment. For example, a working mother may inconspicuously wear the massager at work. To express milk she may take a short break, connect a milk extraction pump and/or activate the massager (optionally this may be done without the need to fully open her shirt and/or remove her bra and/or don a bulky massager). In some embodiments, the massager and/or pump may function discreetly enough that she may continue work during extraction and/or massaging. Alternatively or additionally, the massager may be built into a flange of the pump for milk extraction. For example, the massager and/or collection unit may be designed for easy mounting and/or dismounting to and/or from the breast. In some embodiments, the breast massager may be configured for use with a particular breast pump. Alternatively or additionally the breast massager may be configured to work with a generic and/or a class of breast pumps. Alternatively or additionally, the breast massager may not be associated with a breast pump and/or may be a standalone unit.

### 4 Smart Milk Collection System

An aspect of the current invention relates to a system for collecting milk and/or for recording milk production and/or for controlling distribution of mother's milk and/or for monitoring feeding. For example the system may collect milk and/or measure and/or record production information (for example time, donor [for example the mother] identification, right side/left side, quantity, production rate date time etc.). The system may optionally include a donor unit that may for example package the milk and/or label the packages. For example, packages may be disposable and/or multiuse. For example, the labels may be computer readable. Optionally, the system may include qualitative measurements for example a turbidity measurement and/or a color measurement and/or a fat content measurement. The system may optionally include a receiver unit that for example for verifying that milk is received by the proper recipient (baby), for recording the identity of the recipient and/or the donor, and/or for recording the time and quantity of milk consumed etc. In some embodiments the system may package the milk into packages of a given volume, for example to facilitate control and/or tracking of feeding volume.

In some embodiments, a user interface may be used to give instructions to the user. For example, a view screen and/or touch screen may be provided that supplies user interactive instruction and/or help. Alternatively or additionally, context sensitive instructions may appear on the screen and/or be communicated over a loudspeaker. For example, when the machine is turned on, instruction may be given as to how to put on the retainer and/or massager and/or extraction equipment. For example, when milk production (for example measured as reduction of breast volume) slows, instruction may be given as to how turn up the vacuum and/or end the session. Alternatively or additionally, a user interface may be used for commercial messages. For example in a hospital, at the end of an extraction session the view screen may give instructions as to how to get to the cafeteria to buy a drink and/or at the beginning of a session a user interface may suggest a radio station with relaxing music content. Alternatively or additionally, a regular and/or random suite of advertising may be given over. Alternatively or additionally, user demographic information may be used to select and/or display advertising that is appropriate to the particular user.

In some embodiments, a milk vessel and/or package may include a means for measuring a characteristic of the milk. For example the vessel may include a temperature sensitive coating and/or sticker for indicating the temperature of the milk. For example the coating may change color according to the temperature of the milk. Alternatively or additionally, an indicator may indicate chemical composition of the milk and/or if the milk is still good for eating.

### 5 Faster More Comfortable Milk Recovery

An aspect of some embodiments of the current invention relates to a faster and/or more comfortable breast milk extraction system. For example, milk extraction using a vacuum pump during a breast massaging session and/or a massager according to some embodiments of the current invention may produce milk in 10% to 20% less time and/or 20% to 30% less time and/or 30% to 50% and/or 50% to 75% less time than milk extraction using a vacuum pump without massaging. For example, nursing a baby during a breast massaging session and/or a massager according to some embodiments of the current invention may reduce feeding time by 10% to 20% and/or 20% to 30% and/or 30% to 50% and/or 50% to 75% as compared to nursing without massaging. For example, milk extraction using a vacuum pump during a breast massaging session and/or a massager according to some embodiments of the current invention may produce milk using 10% to 30% lower suction and/or 30% to 50% and/or 50% to 80% lower suction than milk extraction using a vacuum pump without massaging. For example, milk extraction using a vacuum pump during a breast massaging session and/or a massager according to some embodiments of the current invention may produce 10% to 30% more milk and/or 30% to 50% and/or 50% to 80% more milk than milk extraction using a vacuum pump without massaging. For example the average time to empty a breast with massaging may range between 7 to 9 minutes and/or between 9 to 12 minutes and/or between 12 to 15 minutes during a breast massaging session and/or a massager session according to some embodiments of the current invention.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### EXEMPLARY EMBODIMENTS

### 1 A Method

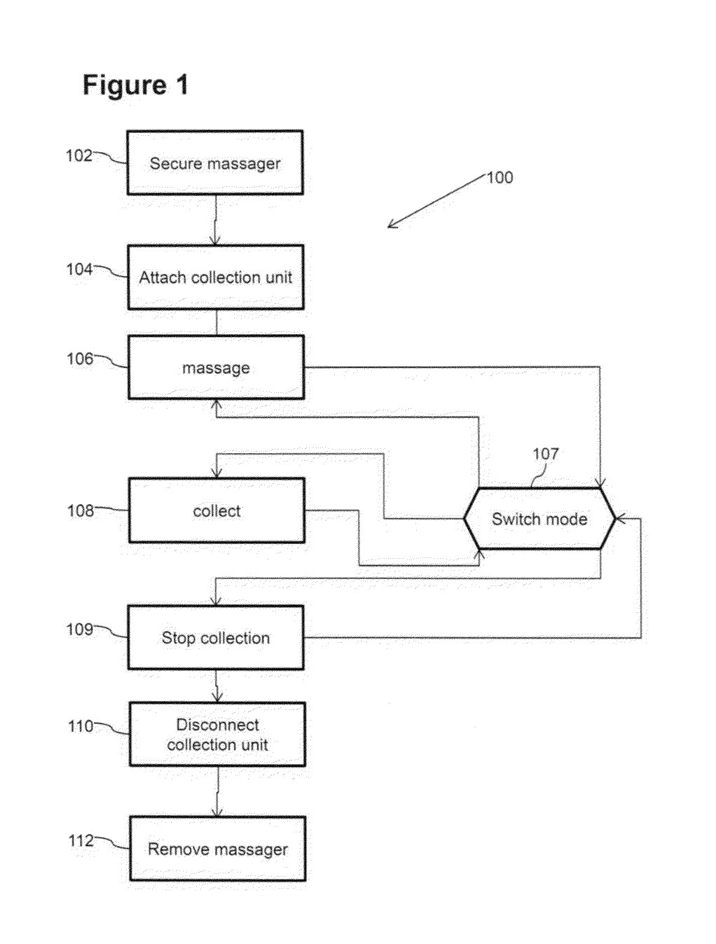

Referring now to the drawings, FIG. 1 is a flow chart illustration of a method of milk extraction in accordance with an embodiment of the present invention. In some embodiments milk extraction may optionally be enhanced by massaging and/or compressing a region of the breast behind the areola including for example a posterior region during extraction. The massaging apparatus may optionally have an inconspicuous design. For example the massager may remain on a woman without disrupting her activities and/or without attracting attention. The extraction system may be designed to function with minimal disruption a woman's activities and/or without attracting attention.

In some embodiments, a woman may put on and/or discreetly secure 102 and/or wear a breast massager when the massager is not in use. For example, when not in use the massager may consist of a few deflated bladders inside a brassiere cup. While deflated, the bladders may be comfortable and inconspicuous. Alternatively or additionally, a massager may be built into a dedicated mounting. For example a massager may be built into a partial bra may be placed over an existing bra. Alternatively or additional a massager may be connected to the breast via an cuff that can be opened and closed, for example like a blood pressure cuff. Alternatively or additionally, a massager may be mounted to the breast with attachments that attach to the existing bra cup and/or straps.

In some embodiments a milk collection unit may allow easy attachment 104 to a breast. Optionally, the massager may be designed to allow placement of a nipple cover (a collection flange) over a nipple. For example the massager may be installed in a brassiere having a front flap that opens and allows placement of a milk collection flange over the nipple without removing the brassiere and/or the massager. Alternatively and/or additionally, the nipple flange may remain on the breast and there may be for example a tubing quick connect protruding through the brassiere for attachment 104 to the collection unit. For the sake of the disclosure the word flange or collection flange may be used to describe a nipple cover. The flange may be hard and/or flexible. In some embodiments the massage unit may massage the areola and/or nipple area from outside the flange, for example by compressing a flexible flange.

In some embodiments massaging 106, and/or collecting 108 may be activated simultaneously. Optionally, massaging 106, and/or collecting 108 may be activated separately and/or serially. In some embodiments, a massager and/or a collecting system may function together or separately, for example as standalone units. Optionally, massaging 106 and/or collecting 108 may be switched 107 between different modes. In some embodiments, a controller may control switching 107. Optionally, switching 107 may change over time according to a program. For example, switching 107 may depend on a time. Alternatively or additionally, switching may depend on an event (for example a change in a measured flow rate of the milk). For example, there may be a first mode with a first manner of massage 106 before collecting 108, for example to encourage relaxation and/or for the production of oxytocin. For example the first mode may last one to five minutes. Additionally or alternatively, the system may then be switched 107 to an optional second mode for example including milk collection 108 and a second different of manner of massaging 106, for example to encourage milk let down. Additionally or alternatively, the system may be switched 107 an optional further manner of massaging 106 later during the collecting 108. For example, switching 107 may occur after a fixed period of time and/or when the milk production rate falls below a certain threshold. For example the later extraction mode may include compression to move remaining milk and/or colostrum and/or high fat hindmilk from the base of the breast towards a nipple. Additionally or alternatively, there may be another switching 107 to a mode including a different manner massage 106 and stopping 109 collection 108. Various programs, modes and/or manners of massaging may for help prevent lactation problems, for example soreness, engorgement, mastitis, and/or blocked ducts. In some embodiments, the manner of the massage (force, cycling etc.) may change. For example, massaging can start softly (less force and/or slower) [for example in order to stimulating the breast to start milk let down]; massaging may optionally continue get stronger (more force and/or faster) for milk expression and/or optionally finish as a different manner of massage (for example softer to comfort and relax the breast and/or the woman).

In some embodiments, massaging may be used to control the quality of milk production. For example, more vigorous massaging and/or massaging of the posterior part of the breast may bring out more hindmilk. The timing of production of hindmilk may be controlled. For example, by massaging the posterior section of the breast in the beginning of extraction, hindmilk may be brought out faster.

In some embodiments, at the end of a collecting session the woman may be disconnected 110 from the collection unit. For example the collection unit may have a flange that covers the woman's nipples. The flange may be held over a nipple and/or areola, for example by a brassiere and/or by vacuum and/or by a massager and/or by hand. The flange may optionally be removed from the nipple and/or tubing may be detached. Optionally disconnecting 110 the collection unit may free a woman to return to regular activities.

In some embodiments, pressure and/or inflation volume may be controlled. For example a pressure transducer may report pressure in one or more rings and/or portions of a ring. A gauge, for example a strain gauge and/or a flow meter (measuring for example the strain and/or volume of squeezing elements) may measure volume changes and/or pressure changes while the breast is being emptied of milk. The pressure control may for example preserve similar pressure on each breast, for example even when the breast size is changing at a differential rate. Optionally measuring the volume and/or pressure simultaneously may facilitate calculating a difference in volume between the beginning and end of the extraction session. The change in volume may indicate of the quantity of milk that was emptied from the breast. In some embodiments monitoring changes of volume and/or pressure in various parts of the breast and/or comparing changes from previous sessions may indicate when and where a problem occurs, for example clogging or infection in the breast.

In some embodiments an inflatable massage element may be inflated until a predetermined pressure value is reached. The time required to inflate the massage element to the predetermined pressure value may be measured. Changes in the time required to inflate the massage element may be tracked over time. Increasing of the time required to inflate a massage element over time may be interpreted to indicate that the volume of inflation of the message element is increasing. Increasing of the volume of filling the massage element may be interpreted to indicate a decrease in volume of the breast. Detecting a decrease in the breast volume may be used to determine whether and/or how much and/or how fast milk is being extracted. Based on changes in inflation, an extraction session may be adjusted. For example, when changes in the breast volume are small and/or the breast volume is not decreasing at a sufficient rate the extraction session may be ended and/or the system may notify a user and/or the user may end the session. The rate of change at which a user is notified and/or a session is ended may include a fixed threshold and/or may include a dynamic value (for example a portion of a measured rate of change at a certain point in the session). Changes in volume may be used to adjust the session in combination with other factors, for example the time of extraction and/or the quantity of milk extracted and/or factors relating to the lactating woman (for example historical milk production data).

In some embodiments massaging and/or measuring volume and/or pressure may shorten the time required to express milk. For example most of the milk may be pumped out in 5 to 7 min. and/or in 7 to 10 min and/or in 10 to 15 min. in some women. Yet it may be desirable for the woman to make sure that the last drops are recovered. For example, the last drops may be considered hindmilk—may include milk that is coming from the roots of the breast-which may sometimes be fatter and/or richer. Compressing the breast may bring the milk from the root of the breast to the nipple and the milk accumulator quicker and allow early cessation of extraction. Alternatively or additionally, hindmilk may include more fat than milk that is extracted earlier. For example, fat may adhere to milk duct tissue. The adhered fat may be removed later in the milking session. For example, the fat content of the extracted milk may increase over time during the milking session. This hindmilk, for example, includes residual milk in the breast, obtained towards the end of an extraction session. For example, hindmilk that remains in the roots and/or base and/or ducts of the breast, may typically include a high fat content. In some embodiments, massaging may improve extraction of residual milk. Improved extraction of residual milk may result in higher fat content in the extracted milk.

Milk quality may remain high and/or rich even for short extraction sessions. For example the rate of milk coming into the collection vessel and/or the rate of change of volume (for example the derivative of the volume) may be calculated. When the flow rate and/or derivative of volume becomes small the system may indicate that to the user that she may want to stop extraction. Measurement may be done also with liquid flow meter or mass meter or weight meter.

In some embodiments, at some time a massager may be removed 112 from the breast. Optionally, the massager may remain on the breast after the collection apparatus has been removed. For example, the massager may optionally be removed 112 at the end of the day before going to sleep. For example, the massager may be built into a brassiere that may optionally be removed 112 upon putting on pajamas before going to bed. Alternatively or additionally, the collection unit may be disconnected 110 and/or the massage unit may be removed 112 at the same time, for example at the end of a milk collection session and/or at the end of the day (for example after multiple collection sessions).

### 2 High Level System Diagram

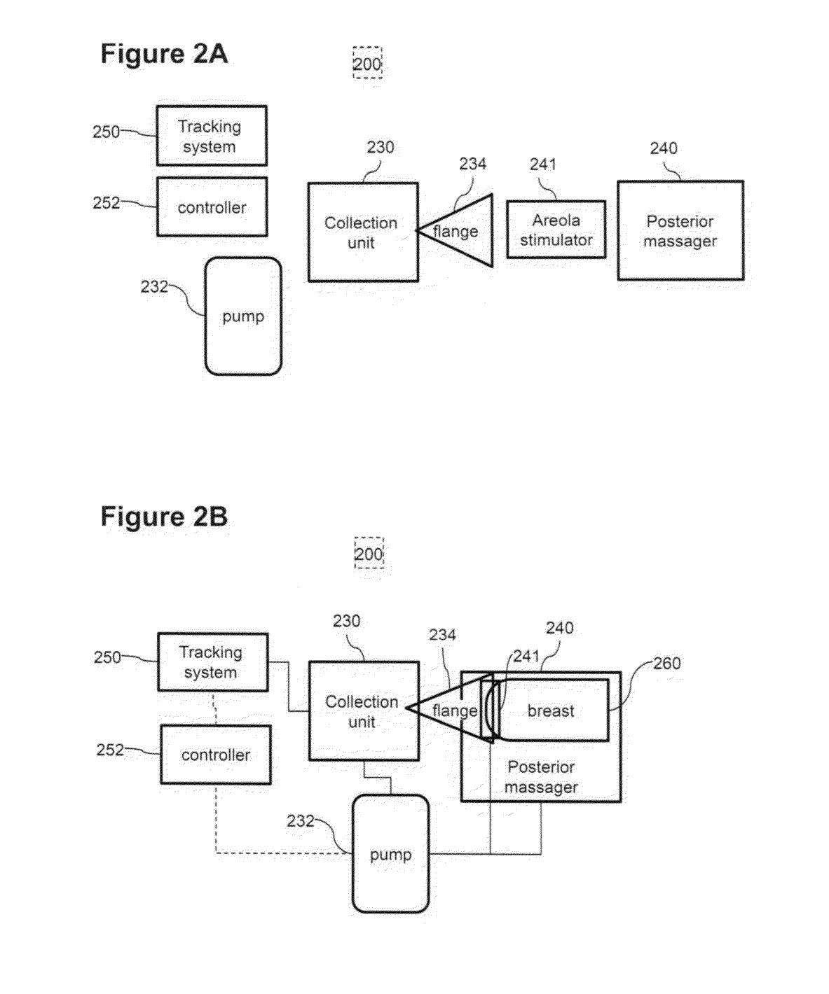

FIGS. 2A, B are high level block diagrams of an exemplary embodiment 200 of milk extraction and/or breast massaging system in accordance with some embodiments of the present invention. Embodiment 200 includes four main sub-systems: a collection unit 230, a posterior massager 240, an areola stimulator 241 and a tracking subsystem 250.

In FIG. 2A the various subsystems are shown separately. Optionally in some embodiments each subsystem (for example collection unit 230, posterior massager 240, areola stimulator 241 and/or tracking subsystem 250) may be a standalone system that may be used on its own or in combination with one or more of the other subsystems of embodiment 200 and/or with an independent device. For example, tracking subsystem 250 may be used to track mother's milk extracted by the extraction system of embodiment 200 and/or by another extraction system (certain options of the tracking system may be available when used with some extraction systems but not others). Optionally, milk may be extracted from the breast into a flange 234. Some embodiments may include a custom flange 234. Alternatively or additionally, the system may be configured to use an existing milk pump and/or flange 234. For example, posterior massager 240 may be used with some or all of the subsystems of embodiment 200 and/or posterior massager 240 may be used in a standalone mode. For example, in standalone mode posterior breast massager may be used for breast enhancement. For example, posterior massager 240 may be used with a conventional breast pump etc. or when breastfeeding a baby. The system may include an engine. For example an engine may include a vacuum pump 232 for example powering collection unit 230. Alternatively or additionally, the engine may include a positive pressure pump, for example powering areola stimulator 241 and/or the posterior massager 240. Optionally, a single engine, for example pump 232, may be used to power multiple subsystems. For example, pump 232 may have a vacuum port for powering collection unit 230 and/or a positive pressure port for powering areola stimulator 241 and/or posterior massager 240. The system may optionally include a controller 252 that may control operation and coordinate operation of one or more subsystems. Alternatively, some or all of the subsystems may include a separate controller and/or engine.

In some embodiments, various systems may be coordinated. For example massaging may be coordinated with milk collecting. For example the controller may activate initial massaging before beginning milk extraction. The controller is optionally programmed to start milk extraction (for example by activating a vacuum pump) and/or change the pattern of massaging after a period of initial massaging and/or without a period of initial messaging. Optionally the user can change the timing, form and/or pressure of the initial and/or subsequent messaging periods. Alternatively or additionally, the controller may turn off, turn on and/or adjust setting of the milk extraction pump and/or massager in response to sensor output (for example based on measured breast volume). During a massaging period, massaging may be constant and/or intermittent. Milk extraction may be performed during a message period. Milk extraction may be constant and/or intermittent. For example, intermittent extraction may optionally be performed during a massaging interval. Alternatively or additionally, extraction may take place during a break between massaging intervals. Optionally, collecting may be coordinated with tracking. For example, a milk collection bag may serve as a collection vessel for the milk. The bag may include a mass sensor, for example a load cell and/or capacitance cell and/or a Wheatstone bridge that senses the tension of the bag. Measurement results of the mass sensor may be transferred over a wire and/or a wireless medium to a controller and/or a tracker. The mass data may be analyzed by the controller and/or logged to a memory. For example flow rate may be calculated. When the rate of flow is small an alarm may sound for example indicating the end of the milking session and/or indicating that the system is not working efficiently and/or the breast is not producing milk.

FIG. 2B illustrates some interconnections of coordinated system according to some embodiments of the current invention. In FIG. 2B the system is illustrated connected to a breast 260. In embodiment 200, controller 252 optionally is a part of tracking subsystem 250 and/or controls pump 232. An engine, for example a pump 232 may in turn optionally power collecting unit 230 and/or areola stimulator 241 and/or posterior massager 240. In some embodiments milk may flow to the collecting unit without adding energy and/or without an engine, for example by free (for example gravitational) flow.

### 3 States

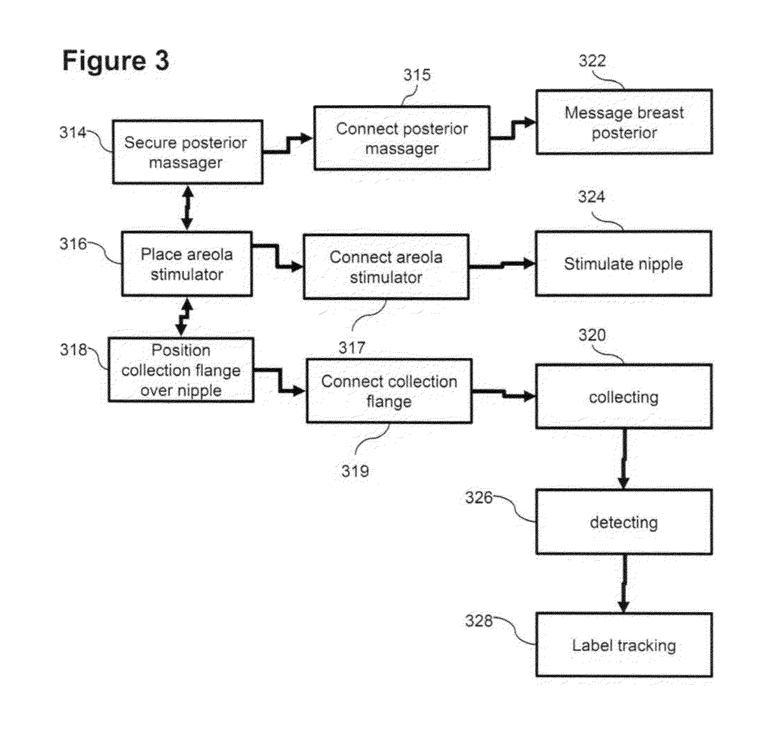

FIG. 3 is a state chart illustrating various states of a milk extraction and/or breast massaging system in accordance with some embodiments of the present invention. The exemplary system of FIG. 3 includes for example three subsystems: a breast posterior massager, an areola stimulator and a collection unit. Each subsystem may optionally be worn and/or activated individually. Alternatively or additionally, multiple subsystems may be worn and/or activated simultaneously and/or in synchronization.

In a first stage of operation, one or more subsystems are initialized. For example, the user may secure 314 a brassiere including actuators of the posterior massager. For example an areola stimulator may be placed 316 onto the breast of the user. For example, a collection flange may be positioned 318 and or held over the nipple of the user. The initialization stage of one or more subsystems may optionally be reversible. Each subsystem may optionally be initiated independently. Alternatively or additionally, initiation of some subsystems may be linked. For example, an areola stimulator may be installed into a collection flange. Positioning 318 the collection flange and placing 316 the areola stimulator may optionally be simultaneous. For example, a brassiere holding a posterior massager may also include a mount for a collection flange. The collection flange may optionally be positioned over the nipple when the massage unit is already being worn.

In some embodiments, when a subsystem has been initiated it may optionally be connected to a controller and/or a power source and/or a collection vessel. For example a posterior massager and/or an areola stimulator may be connected 315, 317 to an engine power source for example a positive pressure pump. A milk collection flange may be connected 319 to a collection vessel (including for example a collection bottle, a collection bag and/or collection tubing) and/or an engine power source (for example a vacuum pump). Alternatively or additionally, some or all subsystems may be initiated (for example secured 314, placed 316 and/or positioned 318) already in a connected 315, 317, 319 state (for example connected to a power source, controller and/or vessel).

In some embodiments a connected subsystem may be reversible activated. For example a power source may activate posterior massager to massage 322 and/or compress the posterior region of a breast. For example, a power source may activate areola stimulator to stimulate 324 an areola and/or a nipple and/or to stimulate 324 milk discharge. For example, a vacuum may be activated continuously and/or periodically to collect 320 milk. As milk is extracted a tracking system may optionally be activated to detect 326 a quantity and/or rate and/or quality of milk production. Milk produced may be packaged and/or labeled and/or tracked 328 (for example when postpartum mother is supplying milk to her newborn baby in a maternity ward of a hospital).

### 4 Coordinated suction flow milk extraction and breast massaging

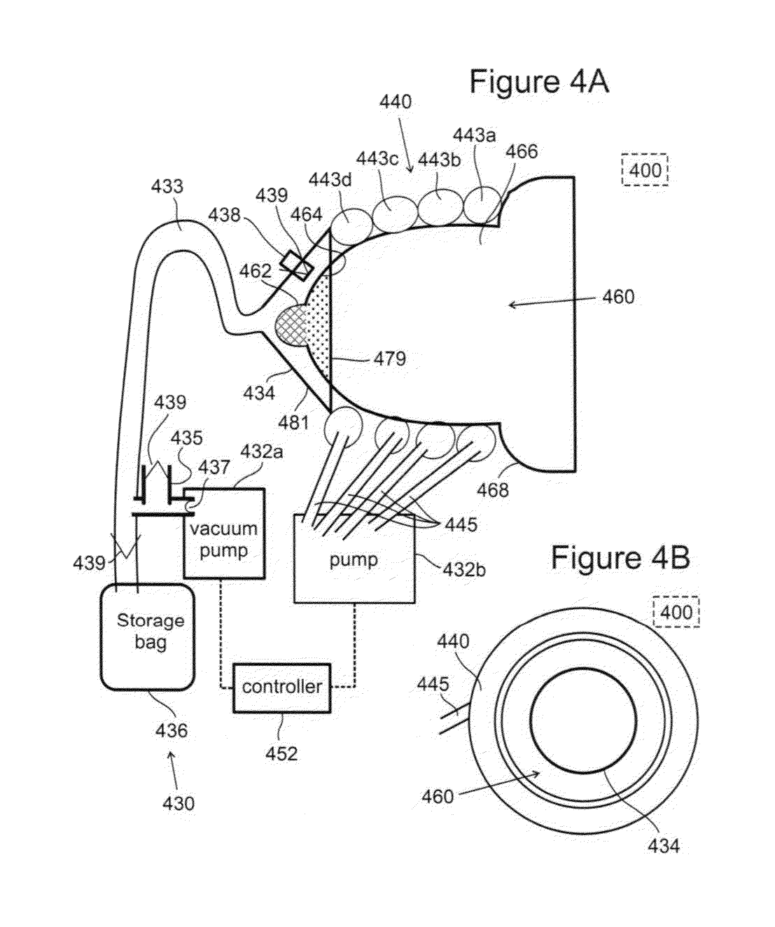

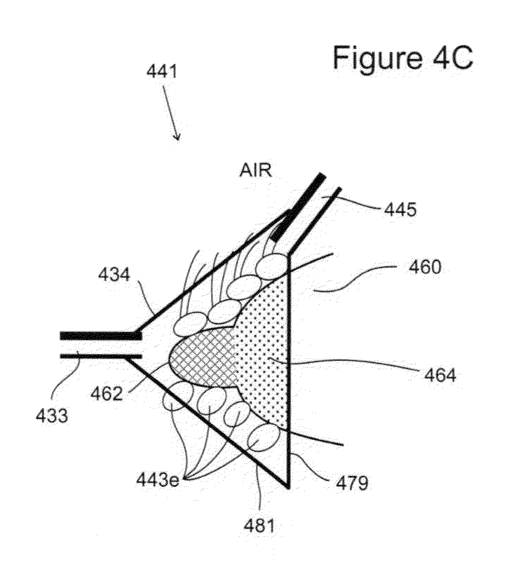

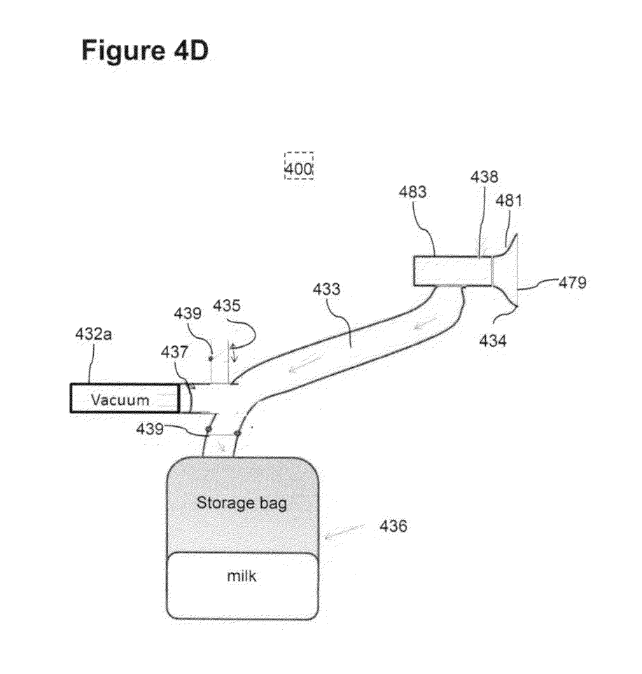

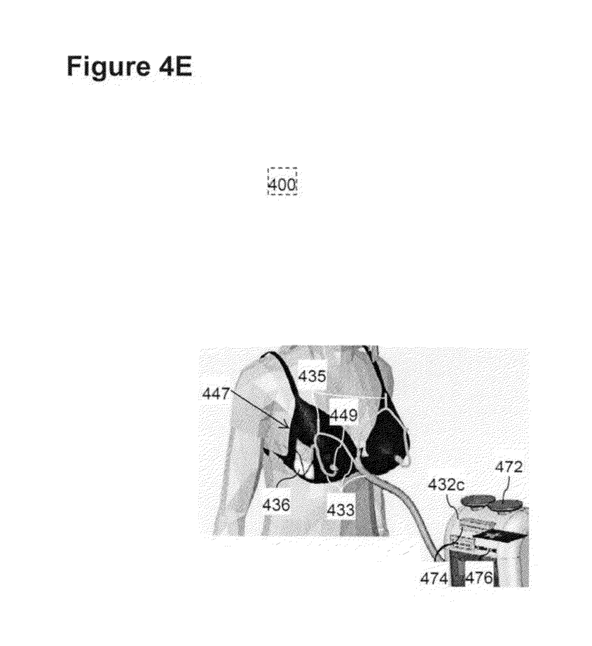

FIGS. 4A, B, C, D, and E illustrate an exemplary first embodiment 400 of a milk extraction system according to the present invention. FIG. 4A illustrates an overall view of embodiment 400. The exemplary system includes, for example, a posterior massager 440 and a milk collecting unit 430. Massager 440 and/or collecting unit 430 may optionally be synchronized and/or operated simultaneously. The system may optionally include an air inlet 438 in a milk collection flange 434. In some embodiments, air flowing into collection flange 434 under vacuum may carry milk through tubing 433. Optionally this may carry milk through tubing 433 even when there is an upward gradient between collection flange 434 and a collection vessel 436.

In embodiment 400, milk may optionally be drawn along tubing 433 from collection flange 434 towards collection vessel 436 via a vacuum. For example collection flange 434 may include an air inlet 438. Optionally, when vacuum pump 432a applies a surge of vacuum, the pressure reduction is passed by a diaphragm 437 to tubing 433 and collection flange 434. Milk is extracted from nipple 462 and into flange 434. For example a breast pump may trigger a milk ejection response and/or letdown. For example, massaging may stimulate let down and/or may push and/or squeeze milk from rear areas of the breast towards and/or out the nipple. Alternatively or additionally, some pumps may optionally achieve this by using suction to pull nipple 462 into the tunnel of flange 434, then release. In some embodiments, alternating air flow may produce a net flow that is practically zero. Alternatively or additionally, air may be sucked into the system through air inlet 438 while milk is being extracted from nipple 462. The air may help dry nipple 462.

Flange 434 may optionally serve as a manifold, mixing air and milk together. Mixing may occur as air is drawn up tubing 433 towards storage vessel 436. An optional pressure release 435 is supplied between diaphragm 437 and collecting flange 434. During pauses in the vacuum, pressure release 435 may release positive pressure oscillations from diaphragm 437. For example pump 432a and/or diaphragm 437 produce positive and negative pressure oscillations; during the negative portion of the oscillation milk is drawn into and/or along tubing 433 towards collection vessel 436; during positive pressure oscillations pressure is released by pressure release 435. The net airflow may be from inlet 438 to outlet 435. In some embodiments the airflow may include an air stream along a collection pathway (for example in tube 433 from flange 434 toward air release 435). Optionally, milk may be entrained into the air stream. For example, the vacuum pump 432a may drive the airstream. Entrainment may include carrying the milk along towards collection vessel 436 by flow of another liquid and/or gas. The geometry of diaphragm 437, pressure release 435, collection vessel 436 and/or tubing 433 may be designed to later separate the milk air mixture. For example, as the milk air mixture approaches diaphragm 437, the milk may fall under gravitation towards collection vessel 436 while air is released through air release 435.

Embodiment 400 optionally includes a posterior massager 440. Massager 440 includes massaging elements 443a,b,c,d which may comprise, for example, inflatable rings encircling breast 460 from the posterior region 466 (for example less than 3 cm from a chest wall 468) until near an areola 464 (for example to within 2 cm of the areola 464). Massaging elements 443a-d are optionally activated by a pump 432b. Optionally massaging elements 443a-d may encircle only a portion of the circumference of the breast. For example, during extraction, pump 432b may optionally periodically apply positive pressure to balloons of elements 443a-d through inflation tubes 445. Optionally pump 432b may pump air, water, oil, and/or some other fluids. Massaging elements 443a-d may optionally be inflated simultaneously, for example to compress breast 460. Breast compression may enhance milk expression. Alternatively or additionally, during milk extraction massaging elements may be used to express milk with a wave like motion. For example, first element 443a may be optionally inflated compressing breast 460 near its base 466 and/or pushing milk from ducts in base 466 towards nipple 462. Subsequently, second element 443b may optionally be inflated compressing breast further pushing milk towards nipple 462. Subsequently, third element 443c may optionally be inflated compressing breast further pushing milk towards nipple 462. While third element 443c is being inflated, element 443a may be released and/or may remain inflated. Subsequently, fourth element 443d may optionally be inflated compressing breast further pushing milk towards nipple 462. While fourth element 443d is being inflated, pressure in element 443b may be released and/or element 443b may remain inflated. In some embodiments, some or all of the rings may be divided into sections that may be controlled separately. Alternatively or additionally, a few rings may be connected, for example in a helical form and/or as multiple connected rings. Alternatively or additionally rings may partially surround the breast, for example a ring may surround more than 70% of the circumference of the breast and/or a ring may surround more than 90% of the circumference of the breast.

In some embodiments, a breast massager may serve additional and/or alternative functions other than enhancing milk extraction. For example a massager may be used to treat engorgement, plugged ducts, and/or mastitis. For example the massager may be used to push fluid in the lymph system out of the breast. Massaging elements 443a-d may optionally be inflated simultaneously, for example to compress breast 460. Breast compression may push fluid in lymph nodes out of the breast. Alternatively or additionally, massaging elements may be used push fluid out of the breast with a wave like motion. For example the wave may be directed from an anterior region of the breast towards a posterior region. For example, first element 443d may be optionally inflated compressing breast 460 near its areola 464 and/or pushing fluid in lymph ducts towards the chest. Subsequently, element 443c may optionally be inflated pushing lymph fluid further towards the chest. Subsequently, element 443b may optionally be inflated pushing lymph fluid further towards the chest. While element 443c is being inflated, element 443d may be released and/or may remain inflated. Subsequently, element 443a may optionally be inflated pushing lymph fluid further towards the chest. While element 443a is being inflated, pressure in element 443c may be released and/or element 443c may remain inflated.

In some embodiments, a local swelling and/or local pain in a breast may be treated by focusing the messaging on the swollen and/or painful location. For example, massaging may include inflation and/or repeated inflation and deflation and/or vibration of a massage element near the affected area. For example the element may include a leaf and/or ring that partially surrounds the breast. Alternatively or additionally, subsonic and/or ultrasonic vibrations may be applied to the area.

In some embodiments, a breast may be massaged to induce labor, to treat the breast for example after surgery, to strengthen breast muscles and/or to shape the breast for aesthetic reasons.

In some embodiments, a controller 452 may control massager 440 and/or coordinate massager 440 and collection unit 430 (for example by controlling pumps 432a, b). One or more one way valves 439 may optionally be supplied. One way valve 439 may optionally ensure that milk flows into but not out of storage vessel 436 and/or to ensure that air enters but does not exit inlet 438 and/or to ensure that air exits but does not enter pressure release 435.

In some embodiments, storage vessel 436 may optionally include a baby bottle. Alternatively or additionally, storage vessel 436 may optionally include a plastic bag. In some embodiment the bag may include structure, for example to prevent the bag from collapsing under the influence of the vacuum in tubes 433. Alternatively or additionally, in some embodiments, one way valve 439 may protect the bag from the effects of the vacuum. The structure of the bag may include an optionally inflatable member. For example, during milk collection the inflatable member may be inflated to prevent collapse, but during storage the inflatable member may be deflated saving space. Inflation of the inflatable member may be manual and/or it may be automatic, for instance by pump 432a and/or 432b.

Optionally, posterior breast massager 440 may be used without milk collection unit 430. For example, massager 440 may be used to for breast compression during baby breastfeeding and/or for stimulating breast growth.

FIG. 4B, illustrates a front end view of embodiment 400 mounted onto breast 460 according to an embodiment of the present invention.

FIG. 4C, illustrates an optional areola stimulator 441 including inflatable ring elements 443e according to an embodiment of the present invention. Elements 443e may be inflated and released via pump 432b and/or an inflation tube 445. Areola stimulator 441 may optionally stimulate nipple 462 and/or areola 464. For example, stimulation may mimic the movements of the tongue and palate of a suckling infant, for example to further encourage lactation, milk let down and/or release. Alternatively or additionally elements 443e of areola stimulator 441 may be positioned on the outside of a flexible flange 434, for example stimulating nipple 462 and/or areola 464 by deforming flange 434.

FIG. 4D illustrates an alternative schematic view of a milk collection system. Parts are marked with the same numbers as corresponding parts of FIG. 4A. In FIG. 4D, there is an air inlet 438 on a connection between tubing 433 and flange 434. Alternatively or additional, an air inlet 438 may be located on the flange. For example, flange 434 may include a breast opening 479, a narrowing portion 481, and/or a nipple channel 483. Optionally, a nipple may be drawn into nipple channel 483 during milk extraction (for example by a vacuum). Air inlet 438 may optionally be located on an anterior portion of nipple channel 483. Alternatively or additionally, an air inlet may be located on posterior portion of nipple channel 483 and/or on tubing 433 and/or on narrowing portion 481. In some embodiments, air flow from air inlet 438 may dry the nipple during milk extraction. Drying the nipple during milk extraction may, in some cases, reduce problems caused by a wet nipple for example chaffing and/or chapping of the nipple.

FIG. 4E illustrates mounting of a milk extraction system (for example embodiment 400) on a woman according to an embodiment of the present invention. In the exemplary embodiment of FIG. 4E, the optional flange, massager and areola stimulator are installed in a brassiere 447. Quick connects 449 on the brassiere allow easy connection and disconnection of a pump 432c to apply suction and/or extract milk. Milk optionally may be collected in convenient single use (and/or reusable and/or sterilized) plastic bag collection vessels 436. For example collection vessel 436 may be held in a pocket in brassiere 447. Optionally a separate collection vessel is supplied for each breast and stored in a pocket in a corresponding cup of brassiere 447. Alternatively or additionally a single collection vessel may collect milk from both breasts. Alternatively or additionally collection vessel 436 may be placed inconspicuously in a cleft between the user's breasts and/or in a pocket of clothing and/or elsewhere. In some embodiments, the combination of massaging and suction may facilitate milk production at reduced suction from conventional milking machines. For example, the suction oscillations may reach a maximum suction ranging between 0 to 50 mmHg and/or between 50 to 70 mmHg and/or between 70 to 90 mmHg and/or between 90 to 120 mmHg and/or between 120 and 200 mmHg and/or more.

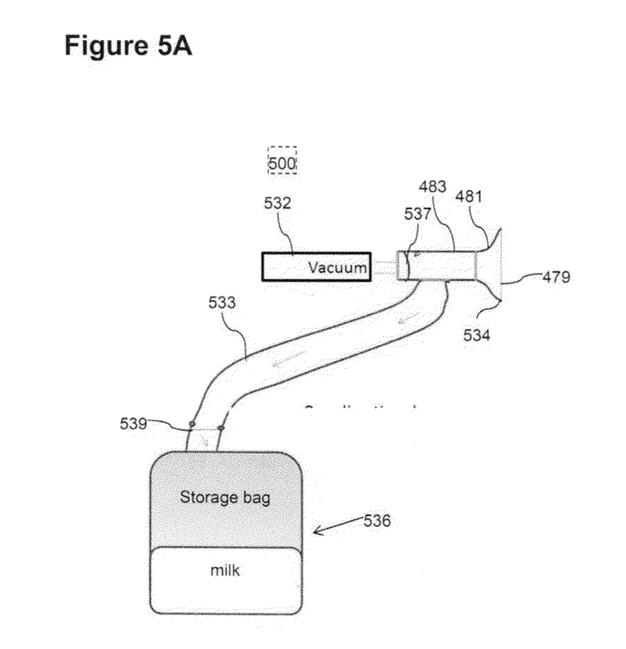

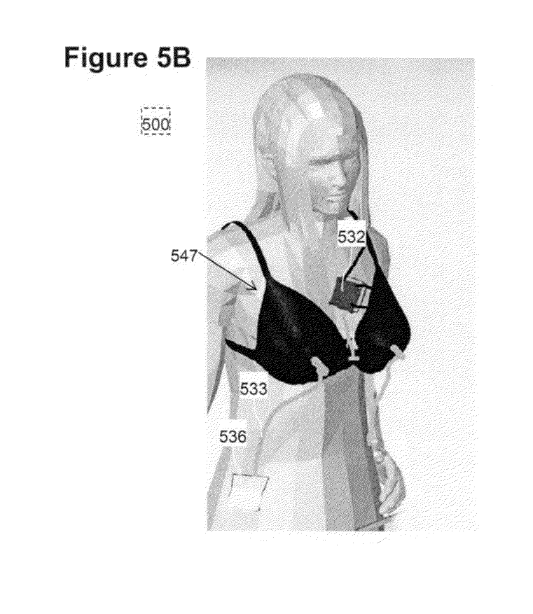

Optionally, pumps 432a, b may include a table standing pump (for example as illustrated in FIG. 4E) which may be powered by an alternating current power grid. Alternative or additionally pumps 432a, b may include a portable battery operated pump (for example as illustrated in FIG. 5B).

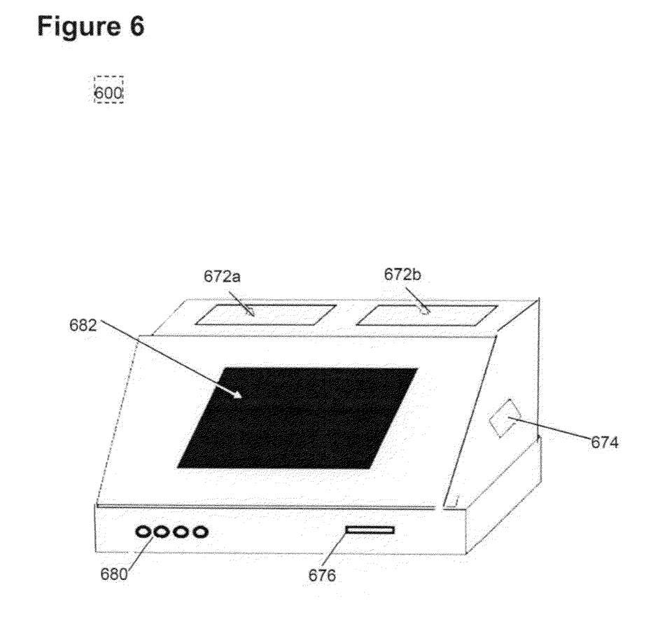

Embodiment 400 may also include an optional tracking system some exemplary aspects of which will be explained hereinbelow with respect to FIG. 6. For example the pump 432c may include a sensor 472 for determining milk output. Alternatively or additional collection vessels 436 may also be supported on a scale (for example a weight sensor 472). The weight of the extracted milk is optionally measured continuously.

In embodiment 400, sensor 472 includes a scale to weight milk output. Alternatively or additionally a strain gauge may be built into vessel 436 and/or tubing 433 and/or a sensor 472 may include for example a turbidity sensor and/or a color sensor to determine a quality of milk output. Optionally, embodiment 400 includes an identifier 474. For example identifier 474 includes a barcode reader and a magnetic strip reader for identifying a bar code on a milk vessel and/or an ID card of a user. Alternatively or additionally an identifier may include for example a fingerprint reader and/or a RFID reader. Pump 432c also includes an optional labeler 476. For example, in embodiment 400, labeler 476 includes a bar code printer that prints bar codes onto stickers used to label milk vessels 436. Alternatively or additionally, a labeler may include a RFID reader/writer and/or a communication device to store identification information on a memory available over a network etc.

### 5 Gravitational Flow Milk Collection

FIG. 5A is a schematic illustration of a gravitation flow milk collection unit according to an embodiment 500 of the present invention. In the gravitational flow unit, there is no inlet for air in collection flange 534. Milk is extracted from the breast by an optional oscillating vacuum and/or diaphragm 537 and/or areola stimulation and/or breast massaging. The milk flows down a tube 533 past an optional one way valve 539 to a collection vessel 536.

FIG. 5B illustrates exemplary embodiment 500 mounted on a woman. Collection vessels 536 for each breast are placed for example on the lower abdomen. Alternatively and/or additionally a single collection vessel may collect milk from both breasts and/or a collection vessel may be held in a pocket of clothing etc. Optionally, a portable battery operated pump 532 is held inconspicuously in a cleavage between the user's breasts. Alternatively or additionally pump 532 may be placed for example in a in a pocket of clothing (for example a special pocket in brassiere 547 and/or a shirt pocket) and/or elsewhere on the user's body. Optionally embodiment 500 may include a table standing pump, for example as illustrated in FIG. 4E of embodiment 400.

### 6 Milk Tracking

FIG. 6 illustrates a milk tracking unit according to an embodiment 600 of the present invention. A milk tracking unit may track quality and/or quantity of milk output. The milk tracking unit may also be used at a baby feeding station to track the quantity and quality of milk consumed by a baby and/or to verify that the baby is receiving the proper milk (for example milk from his own mother). A milk tracking unit may be a standalone unit as illustrated for example in embodiment 600 and/or be built into another device, for example a pump (for example as illustrated in FIG. 4E).

Embodiment 600 may include, for example, a sensors 672a, b for determining milk output of a left and right breast respectively. In embodiment 600, sensors 672a, b include a hollow into which the user places the full milk bag after extraction. The hollow may include for example a scale to weight milk output and/or a turbidity sensor and/or a color sensor to determine a quality of milk output. Optionally, embodiment 600 includes an identifier 674. For example identifier 674 includes a barcode reader for identifying a bar code on a milk vessel and/or an ID card of a user. Alternatively or additionally an identifier may include for example a magnetic strip reader and/or fingerprint reader and/or a RFID reader. Embodiment 600 may also include an optional labeler. For example, in embodiment 600, labeler 676 includes a communication port to store and/or retrieve identification information on a memory available over a network and an RFID read/write unit (not shown). Alternatively or additionally, a labeler may include for example a bar code printer that prints bar codes onto stickers used to label milk vessels.

Embodiment 600 of a milk tracking system also includes an optional user interface. For example a user interface may include an input interface 680, for example buttons. Alternatively, an input device could include a hard wired or wireless device for example a mouse, a keyboard and/or a mobile communication device (for example a cell phone). For example, a user interface may include an output interface 682, for example including a display screen. In some embodiments the output interface may be used to encourage lactation, for example by showing pictures of the user's baby, another baby and/or by playing relaxing music and/or baby sounds. Alternatively or additionally, the user interface may include a dual purpose (input/output) device such as, for example, a touch screen. Alternatively or additionally, the user interface may include a loudspeaker and/or a microphone etc.

### 7 Collection Bag

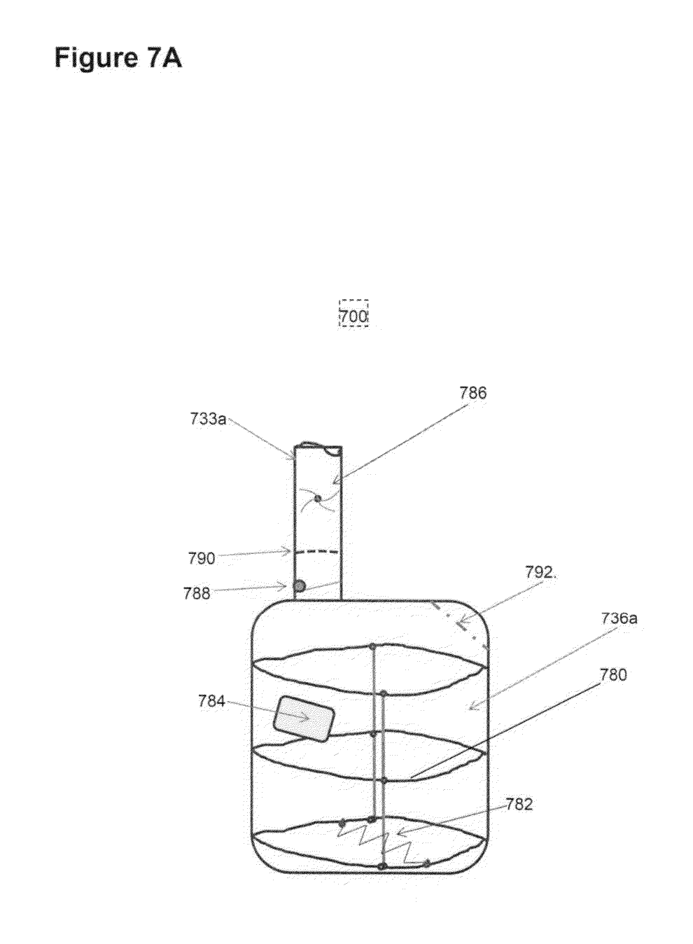

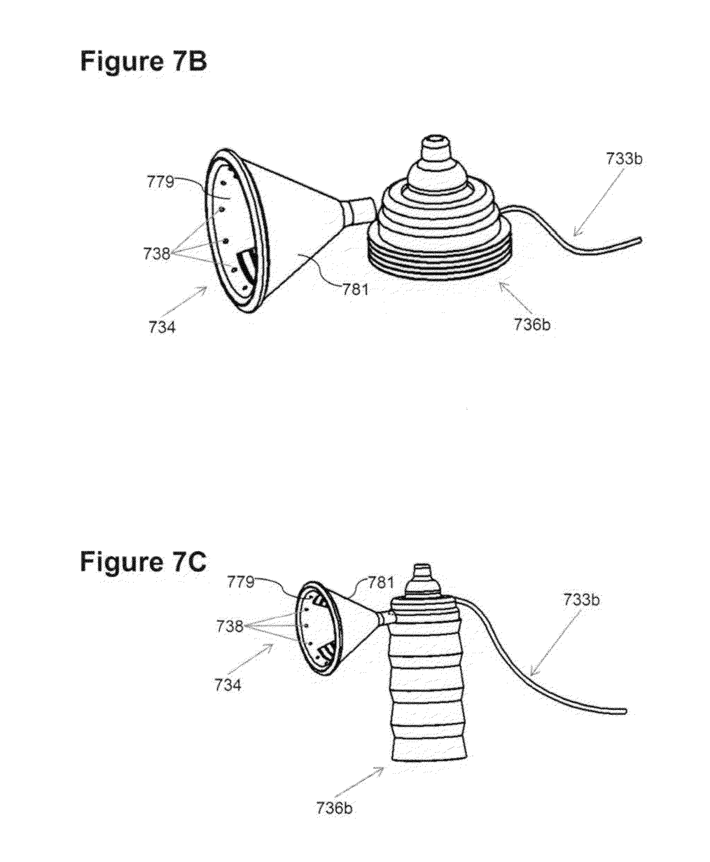

FIG. 7A illustrates an exemplary collection vessel 736a according to an embodiment 700 of the current invention. For example collection vessel 736a includes for example a flexible single use bag. Optionally, the bag may be collapsed, for example for easy storage and/or transport of empty bags. Optionally the bag may be expanded for example for holding milk.

In some embodiments, collection vessel 736a may have a structure, for example holding the bag in the expanding configuration under vacuum. For example in embodiment 700 the structure includes a wire frame 780. Optionally, frame 780 may be stiff enough to hold the bag in the expanded configuration under the vacuum of a milk pump, but under full vacuum the bag may collapse. For example the bag may be sold, stored and/or transported vacuum sealed and/or in a collapsed state and/or may spring into the expanded configuration, ready for use, when the bag is initially unsealed. Alternatively or additionally, frame 780 may include inflatable members that may be collapsed or expanded for example by inflation and/or deflation at the convenience of the user. Optionally a frame 780 may include a tension member. For example a spring 782, may control expansion of vessel 736a.

In some embodiments, milk vessel 736a may include an input tube 733a. Input tube 733a may optionally include a one way valve 788. One way valve 788 may prevent collapse of the bag under the vacuum of a breast pump and/or prevent leak of milk when the bag is detached from the collection system. For example, there may be a seal and/or cutting line 790 for sealing vessel 736a and/or to detach the bag from the collection system at the end of a milking session and/or when the bag is full. Optionally, a collection vessel 736a may be single use. For example, there may be a cutting line 792 for opening vessel and/or emptying its contents for use.

In some embodiments, vessel 736a may include one or more sensors. For example a strain gauge 784 may be connected to the walls of vessel 736a. Measurements of the stress and/or strain on the walls of vessel 736a may facilitate estimation of the mass and/or volume of the contents. Alternatively or additionally, a strain gauge may be attached to frame 780 for example to spring 782. Optionally, measurements may be recorded on a memory internal to vessel 736a and may be read by a reader. Alternatively or additionally there may be a transmitter powered by a miniature battery and/or an external stimulation (like for example an RFID) to report the strain and/or milk content. Alternatively or additionally, vessel 736a may include a chemical detector. For example a color coded strip may measure pH and/or oxygenation state of bag contents. For example, the pH of the milk may be measured by a color detector outside the bag. Alternatively or additionally vessel 736a may include a flow meter, for example a turbine meter 786 measuring, for example, an input flow rate.

FIGS. 7B and 7C illustrate a collapsible milk vessel 736b according to an embodiment of the current invention in a collapsed and expanded configuration respectively. Vessel 736b may for example be shipped and stored in a collapsed state and/or used for milk collection and/or storage in an expanded configuration. Milk vessel may optionally include a stiffener element (for example a post [not shown]) to hold it in the expanded configuration under vacuum. Vessel 736b is illustrated connected to an optional milk collection flange 734. Flange 734 may optionally serve as a manifold mixing milk and air. For example, air may enter flange 734 via one or more air inlets 738. For example air inlets 738 may be located on a narrowing portion 781 near a breast opening 779 of flange 734. A vacuum may be applied to vessel 736b via a tube 733b.

### 8 Flange

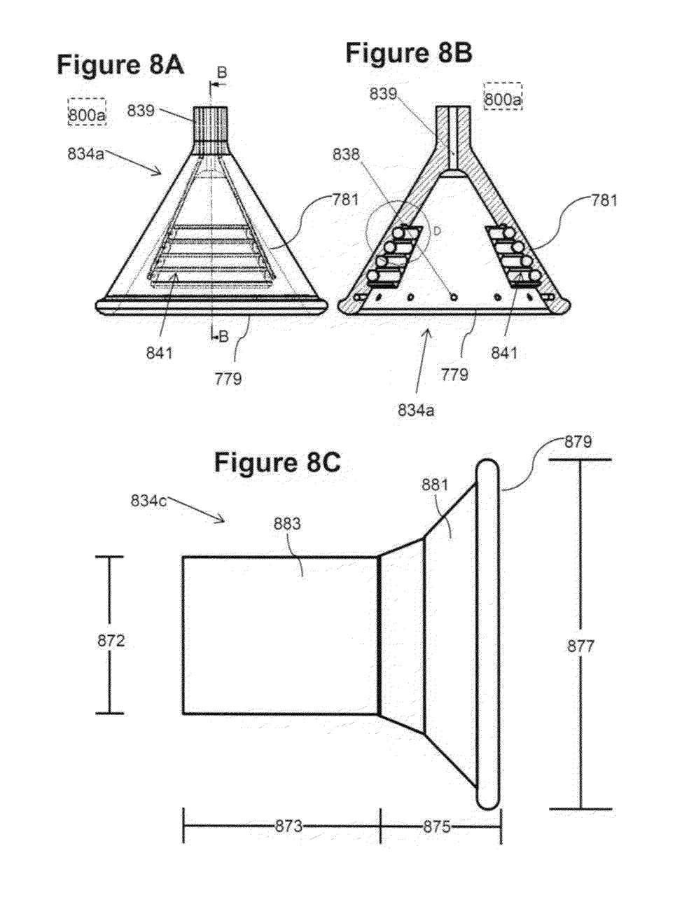

FIGS. 8A, B illustrate an exemplary embodiment 800 of a collection flange 834a with a built in areola stimulator 841 in accordance with an embodiment of the current invention. Flange 834a includes optional air inlets 838. With air inlets 838, flange 834a may act as a manifold to mix air and milk and facilitate vacuum suction transport of collected milk for example as explained above with regards to FIG. 4A. In the exemplary embodiment 800, optional air inlets 838 may be located for example at the posterior end of flange 834a.

Flange 834a may include an optionally connector 839. For example connector 839 may be a standard breast pump connector. Flange 834a may be compatible with many models of breast pumps. Alternatively or additionally, flange 834a may include a custom connector for use with a dedicated breast pump.

Areola stimulator 841 may include for example multiple inflatable rings. Rings may optionally encircle a nipple and/or an areola. Rings may be inflated serially and/or simultaneously. For example the stimulation may mimic stimulation of the nipple and/or areola by the palette and/or tongue of an infant during breast feeding. Areola stimulator 841 may express milk on its own and/or may be used in combination with a vacuum pump to extract milk. Optionally, areola stimulator 841 may be used without an extraction unit for example to help induce and/or accelerate labor.

FIG. 8C is a schematic drawing illustrated dimensions of a flange 834c of a milk extraction system in accordance with some embodiments of the present invention. Flange 834c may be used with various embodiments of the current invention for example in the embodiments described herein above and below. For example the width 877 (for example the diameter) of the breast opening 879 of flange 834c may range between 35 to 55 mm and/or between 55 to 65 mm and/or between 65 to 80 mm. In some embodiments, a flange held to a breast by a retainer may have a smaller breast opening than standard breast flanges (for example the smaller opening may be easier to fit through an opening of the retainer and/or because the retainer holds the flange to the breast, the flange may be more stable with a smaller opening). The length 875 of narrowing portion 881 may range, for example between 10 to 20 mm and/or between 20 to 40 mm. The length 873 of a nipple channel 883 of flange 843 may range for example between 5 to 15 mm and/or between 15 to 30 mm and/or between 30 to 50 mm and/or larger. For a flange 834 used with a breast massager, nipple channel 883 may be shorter than a standard collection flange. For example, massaging may facilitate extraction with reduced vacuum (as explained herein above and below). The nipple will optionally be pulled less deeply into nipple channel 883. The width 872 (for example the diameter) of nipple channel 883 may range for example between 10 to 20 mm and/or between 20 to 30 mm.

### 9 Alternative Embodiments of Breast Posterior Massager

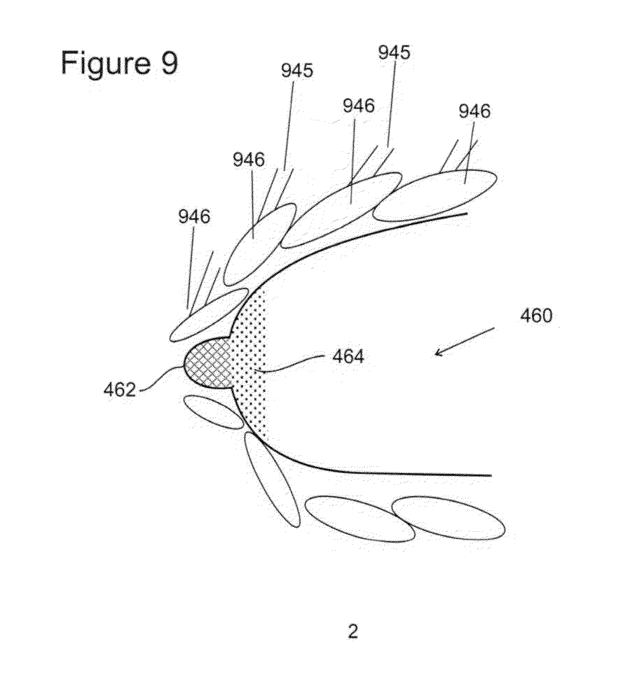

FIG. 9 illustrates an alternative embodiment 900 of a posterior breast massager according to an embodiment of the present invention. For example embodiment 900 includes overlapping inflatable leaves 943. For example each leaf 943 overlaps adjacent leaves 943. Overlapping leaves may reduce the gap between massaging elements in comparison to round tubes. In some embodiments, leaves 943 may each encircle breast 960 and/or alternatively or additionally leaves 943 may be tiled over breast 960, each leaf covering a small portion of the circumference of breast 960. In some embodiments, leaves 943 may be inflated individually and/or in groups by means of inflation tubes 945. For example, one or a few leaves may be activated to massage (for example to squeeze and/or to alternatingly squeeze and release and/or to vibrate) a particular spot on the breast. For example a massage may be applied to a painful and/or hard spot on the breast, for example in the case where the spot is a due to blocked duct.

Squeezing and/or massaging may be for the whole breast and/or a portion thereof. For example massaging and/or squeezing may cover a third of the breast or more. For example the massaging may be applied to an anterior portion of the breast and/or a posterior portion. Inflating may optionally be continuous. For example in a manner similar to inflating a long balloon when it starts inflating in one point and then from that point it goes on.

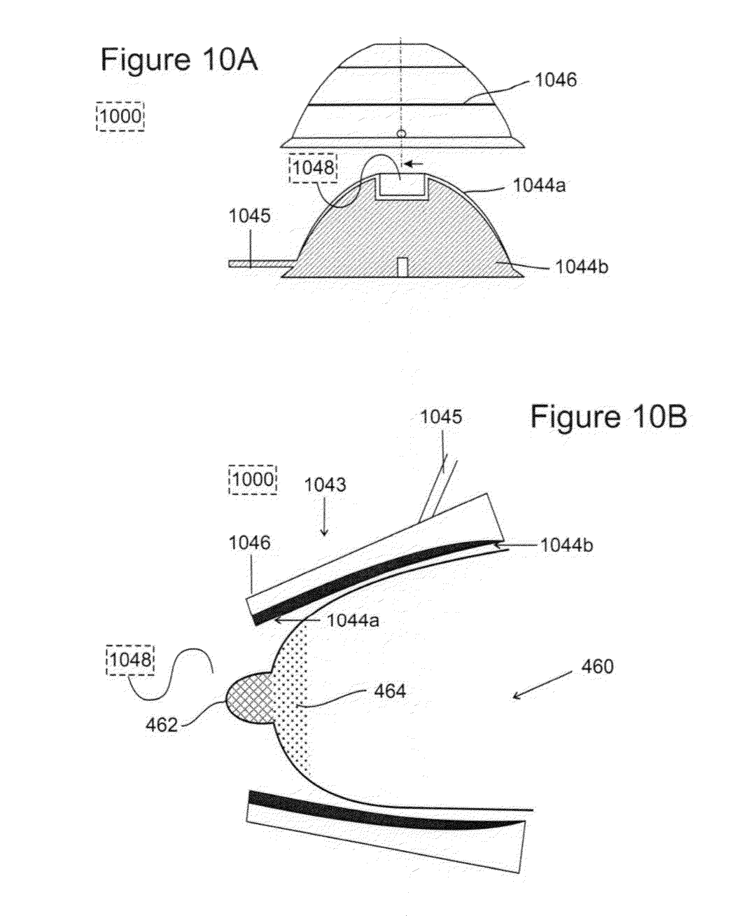

FIG. 10A, B illustrate a posterior breast massager according to an alternate embodiment 1000 of the current invention. Optionally a messaging element 1043 is constructed of an external relatively inelastic shell 1046 and an elastic inner wall having with increasing thickness from posterior end 1044b to an anterior end 1044a. When air or liquid is pumped into outer shell 1046 via a tube 1045 the inner wall extends inward squeezing the breast 460. At first the thinner posterior end 1044b extends squeezing the posterior end of breast 460 and as pressure increases extension continues forward to thicker and thicker portions until it reaches the anterior end 1044a. Optionally, the inner wall may have larger surface than outer shell 1046. An opening 1048 may optionally be supplied for exposing an areola 464 and a nipple 462. Optionally a baby may suckle from nipple 462 protruding through opening 1048 and/or a flange of a milk collection unit may be placed through opening 1048 over nipple 462.

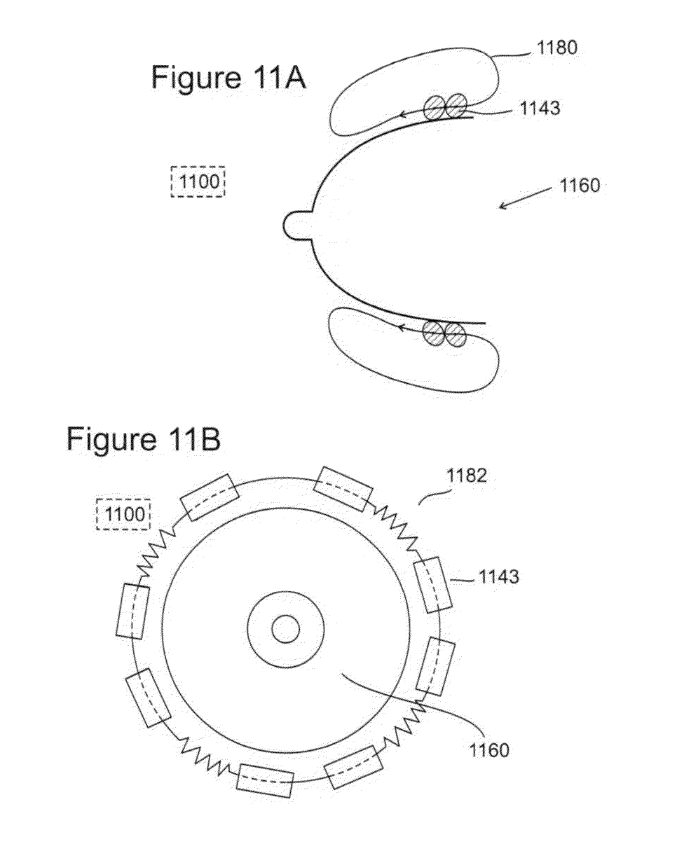

FIG. 11 illustrates a mechanical breast massager according to an alternative embodiment 1100 of the current invention. Optionally squeezing of a breast may be achieved mechanically with non-inflatable elements. For example in embodiment 1100 massager 1140 includes wheels and/or rollers 1143 that roll over and massage the breast.

In some embodiments, the rollers 1143 may travel on a path 1180. As rollers 1143 travel down the first leg of the path they may optionally squeeze the breast 1160 from its posterior end to its anterior end (towards the areola 464). The rollers may then return on a return leg of path 1180 without contacting the breast. Once completing the return leg rollers 1143 may again travel down the first leg squeezing breast 1160. Rollers may be connected by an elastic member 1182, for example a spring and/or an elastomer. Although the diameter of the breast may change along its length, the squeezing force of the rollers (supplied by the elastic) may optionally be maintained by elastic element 1182.

### 10 Fitting to Breast

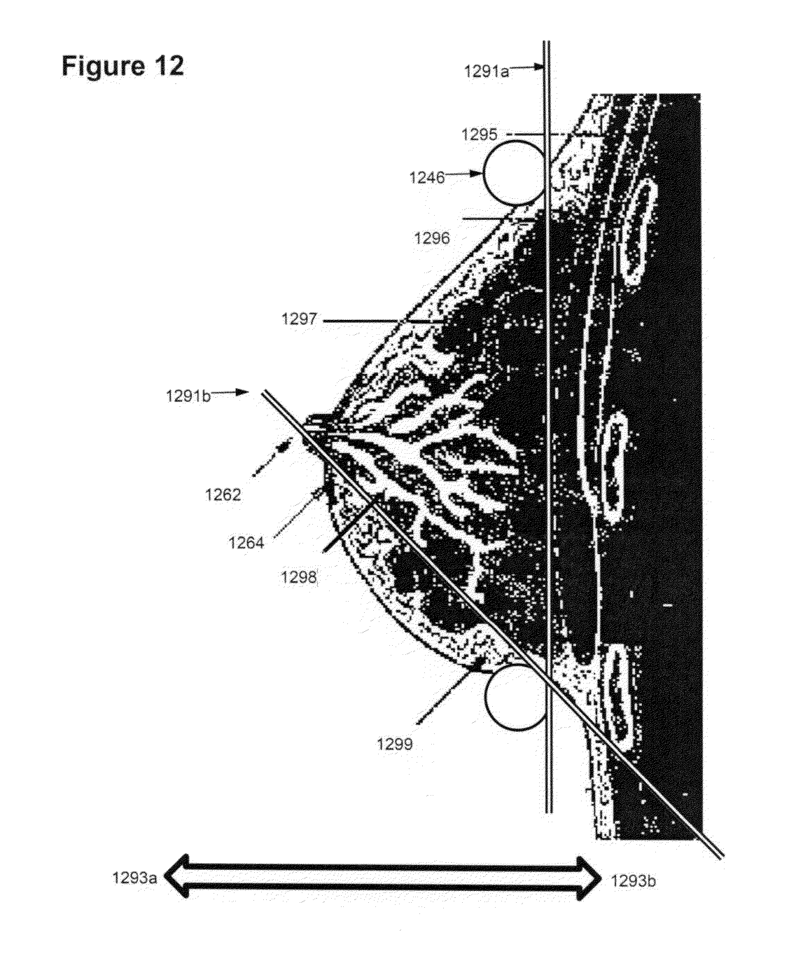

FIG. 12 is a schematic depiction of breast anatomy and a pneumatic massage element in accordance with an embodiment of the present invention (image of breast anatomy was supplied from SEER Training Modules, Module Cancer surveillance and training—Breast cancer—Anatomy. U. S. National Institutes of Health, National Cancer Institute 27 Aug. 2013). In the drawing various elements of breast structure are marked including: a chest wall 1295, pectoralis muscles 1296, lobules 1297, ducts 1298, and fatty tissue 1299. An anterior direction 1293a (towards the front of the woman, towards the nipple 1262 of the breast, leftward in the FIG. 12) and a posterior direction 1293b (towards the back of the women, in the direction of the chest wall 1295 from the breast, rightward in FIG. 12) are illustrated by a block arrow. The posterior of a massage element (for example pneumatic message element 1246) may for example be posterior to 95% of lobules 1297 and/or ducts 1298 and/or volume of the breast (for example 95% of lobules 1279 may be anterior to a plane posterior to all the massage elements [for example a plane passing through line 1291a and one other point for example on element 1256 where it contacts a side of the breast]). Alternatively or additionally the posterior of the massage element may for example be posterior to 85% of lobules 1297 and/or ducts 1298 and/or volume of the breast. Alternatively or additionally the posterior of the massage element may for example be posterior to 70% of lobules 1297 and/or ducts 1298 and/or volume of the breast. Alternatively or additionally the posterior of the massage element may for example be posterior to 50% of lobules 1297 and/or ducts 1298 and/or volume of the breast. For example, the posterior of element 1246 is illustrated by line 1291a. Alternatively or additionally, the posterior point on the lower portion of massage element 1246 on a vertical cross section that passes the most anterior point of the breast (for example the tip of nipple 1262) may be posterior for example to 80% of a line that passes through and the most anterior point of the breast and the most posterior point on the lower half of massage element (for example line 1291b). Alternatively or additionally, the posterior point on the lower portion of massage element 1246 on a vertical cross section that passes the most anterior point of the breast may be posterior for example to 50% of line 1291b.

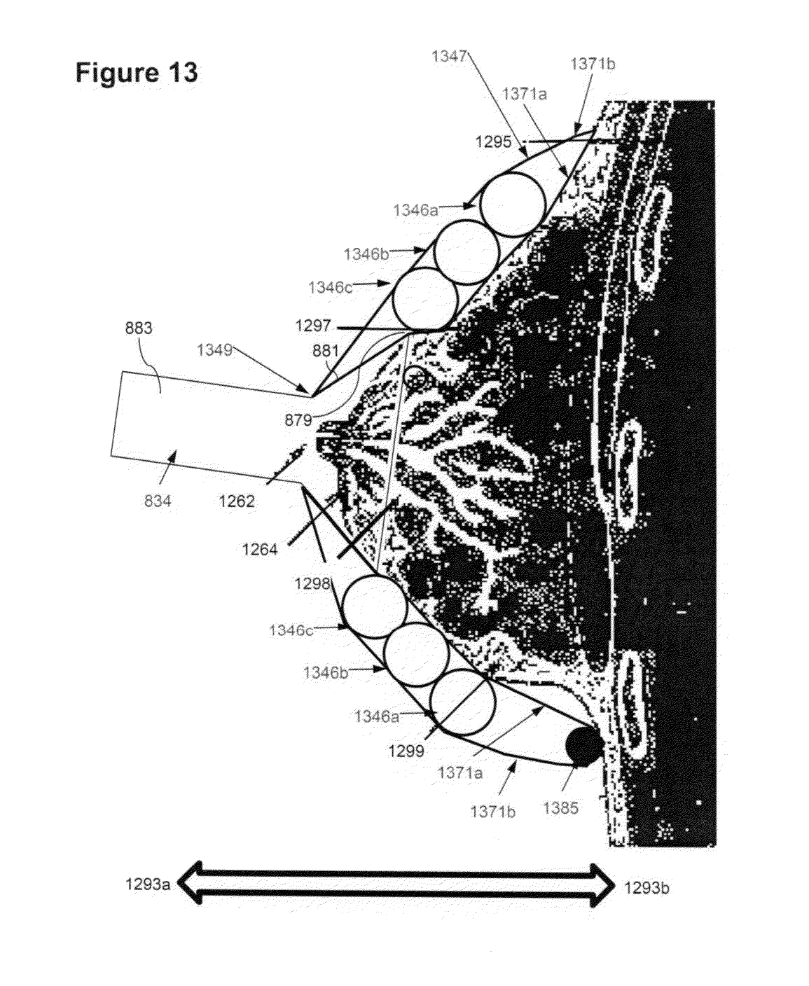

FIG. 13 illustrates a milk extraction system mounted on a breast in accordance with an embodiment of the current invention. The milk extraction system includes a flange 834 positioned over a nipple 1262 to collect milk. In some embodiments flange 834 may be held in place by a retainer 1347. Flange 834 is optionally inserted through an opening 1349 in retainer 1347. Optionally, retainer 1347 also secures a massager to a posterior region of the breast. For example, in the embodiment of FIG. 13, three pneumatic massage elements 1346a, 1346b and 1346c are secured around the breast starting from an edge of breast opening 879 of flange 834. For example, elements 1346a-c are annular elements surrounding the breast and deployed along the breast behind (in the posterior direction 1234b from) breast opening 879 of flange 834. In some embodiments a massager may include two and/or four and/or more massage elements. For example, a massager for a smaller breast may include fewer elements and/or a massager for a larger breast may include more elements. Alternatively or additionally, a massager for a larger breast may include larger massage elements and/or a massager for a smaller breast may include smaller elements.

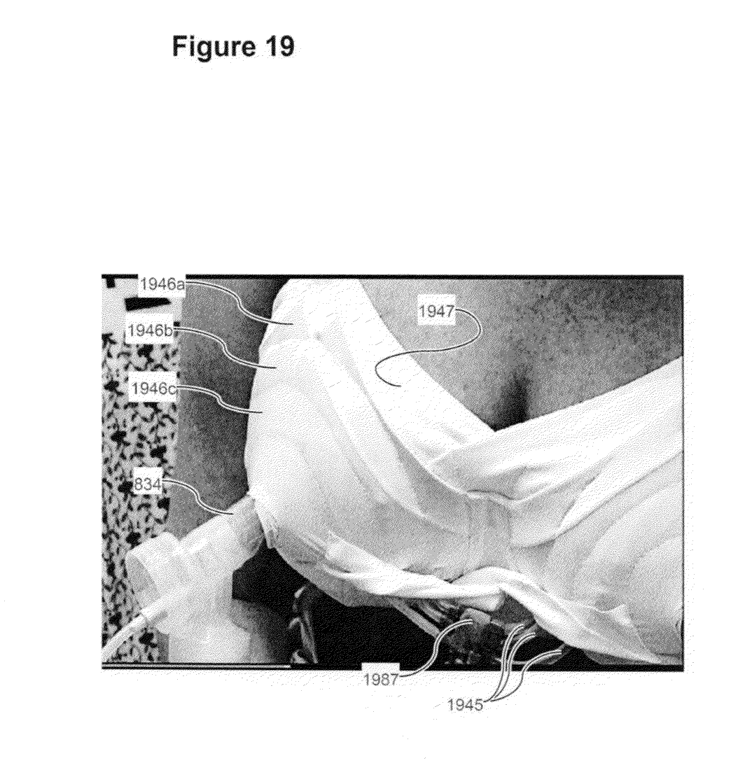

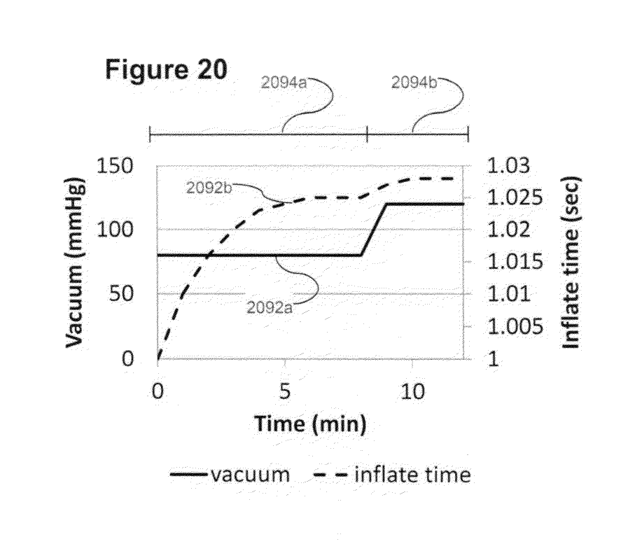

In some embodiments, massage elements 1346a-c have an annular shape. For example each massage element 1346a-c may have the form of a ring torus. For example each ring element 1346a-c may have a minor radius ranging between 0.2 to 1.0 cm and/or between 1 to 3 cm and/or greater than 3 cm. For example, in FIG. 13, each ring has a minor radius of 1.0 cm. For example, in FIG. 13, third ring element 1346a massages an annular region centered 3 cm from the edge of flange 834 and the second ring element 1346b massages annular region centered 1.5 cm from the edge of flange 834 and the first ring element 1346c annular region centered 0.5 cm from the edge of flange 834. In some embodiments a massager may include more or fewer elements. The elements may optionally be larger or smaller than the exemplary elements of FIG. 13. For example in FIG. 19 each ring has a minor radius of 2 cm. Alternatively or additionally, a breast massager may include multiple massage elements, each element having a different radius. For example, when opened to a cylindrical shape a message element may have radius (minor radius of the torus) as described above and/or a length of between 400 and 800 cm. The inflation volume of each message element may range, for example, between 5 to 15 liters and/or between 15 to 25 liters and/or greater than 25 liters.

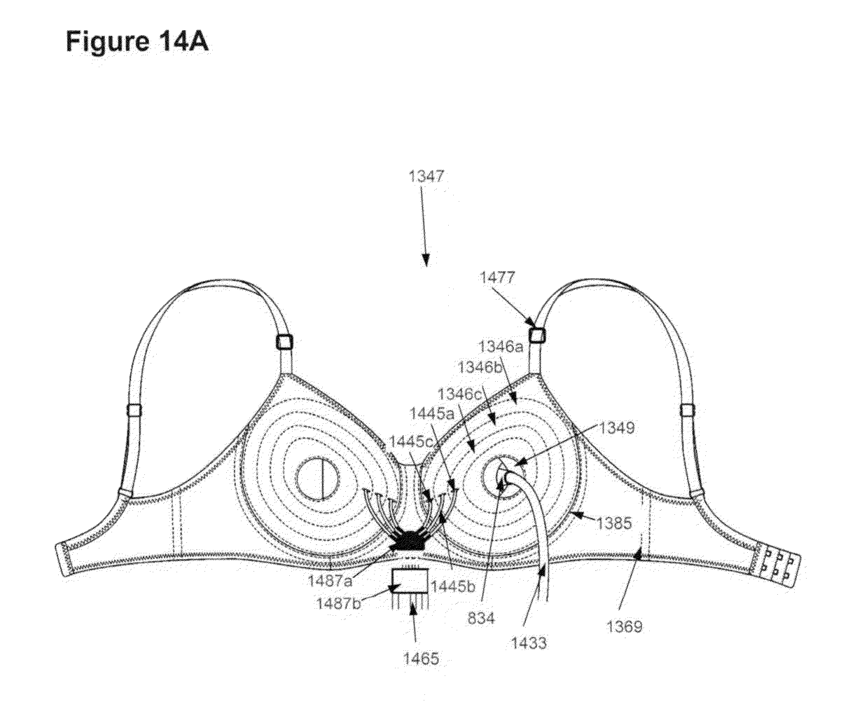

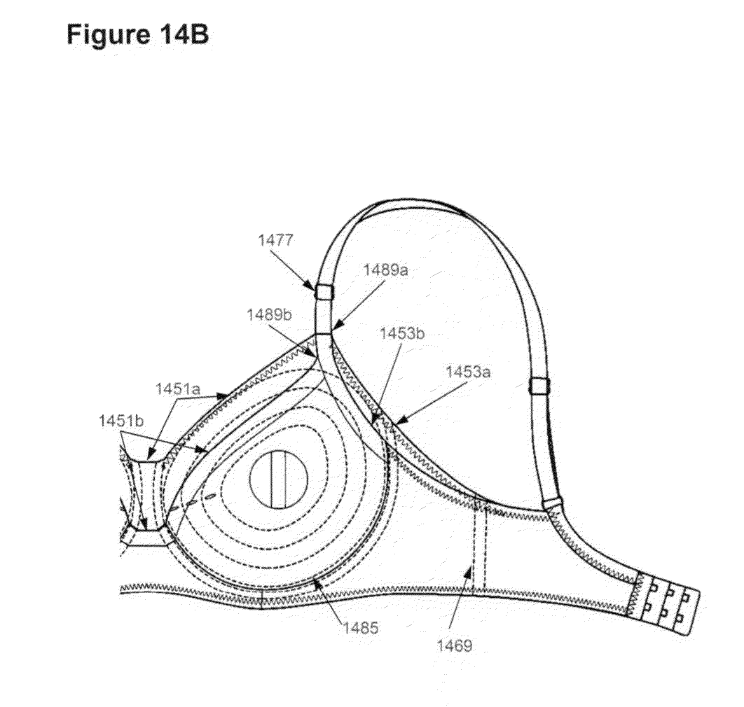

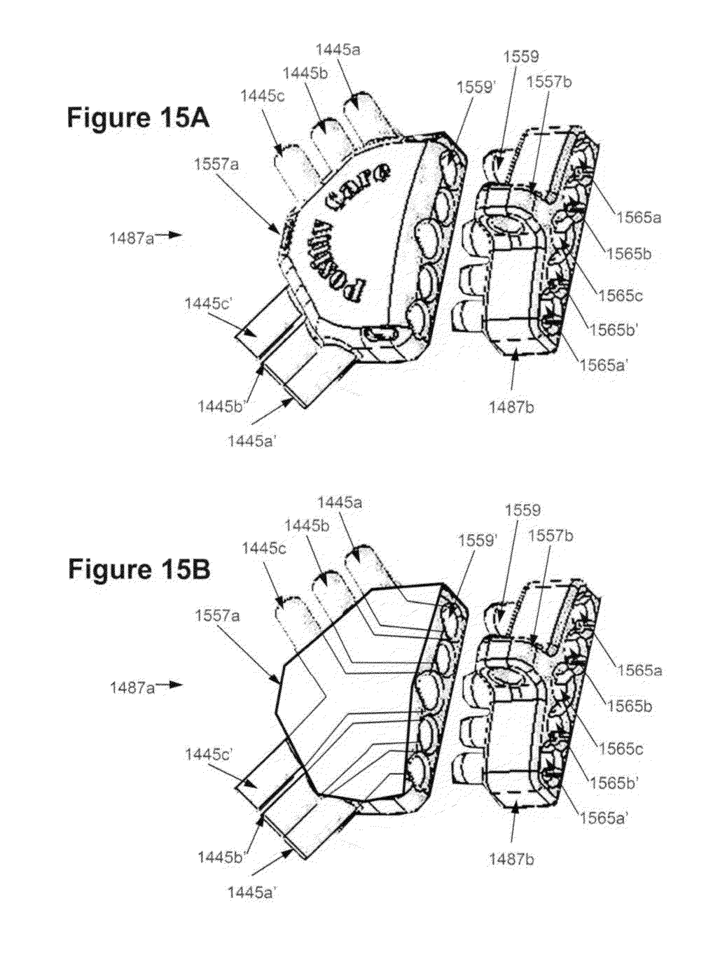

In some embodiments, retainer 1347 includes, for example a brassier (for example as illustrated in FIGS. 14A and 15). Retainer may be formed of two layers, for example an inner layer 1371a and an outer layer 1371b. Optionally, inner layer 1371a is more elastic than outer layer 1371b. For example, when elements 1346a-c inflate and/or expand, they may be prevented from significant outward expansion by outer layer 1371b and/or may expand inward stretching inner layer 1371b. Optionally, the inward expansion compresses the breast. In some embodiments, when fully inflated, the message elements may distort to fill the space in the retainer, for example similar to an inner tube filling a tire.

Outer layer 1371b optionally includes pleats or other structures that let it expand in a chosen direction to form the breast in a preferred geometry. For example, pleats on the bottom of the retainer 1347 may allow the breast to expand outward and/or upward and/or radial pleats may allow the breast to expand outward into a bullet shape. The outward expansion may for example take weight off the bottom of the breast and/or reduce folding of the bottom of the breast and/or open milk pathways in the bottom of the breast.

In some embodiments a massager may be designed to clear a blockage to milk flow. For example, the massager may be made to clear a blocked milk duct. For example the massager may be designed to clear blockages in the bottom of the breast. Balloons under the breast may optionally support and/or reduce folding in the lower part of the breast. In some embodiments massage elements may be supplied under the breast but may not surround the breast on all sides.

In some embodiments, the lower portion of retainer 1347 may is held in place by an underwire 1385. For example, an underwire may be made of metal and/or nylon and/or another material. Alternatively or additionally a bottom wire may pass along the bottom of the retainer. A bottom wire may for example hold the bottom of the retainer to the chest wall of the user. Optionally a cross wire may run from the upper strap of the retainer to the bottom of the retainer (for example attaching to a bottom wire). A cross wire may optionally help to hold the center of the retainer (between the breasts) to the chest wall.

### 11 Brassier Type Retainer

FIG. 14A illustrates a front view of a retainer for massaging and/or breast milk extraction according to some embodiments of the current invention. Optionally, the retainer has the form of a brassier. Optionally, the retainer serves as a brassier for normal use and/or is suitable for retaining a milk collecting and/or a breast massaging system when needed. For example, messaging elements and/or adaptors and/or connectors for the milk collection and/or massaging system may be designed to be unobtrusive when not in use. For example, retainer 1347 of FIG. 14A includes optional pneumatic message elements 1346a-c that are unobtrusive when not inflated. For example, retainer 1347 includes an optional opening 1349 allowing insertion and/or removal of a milk collection flange 834 without removal of the brassier. For example, retainer 1347 includes a tubing connector 1487a that allows connection and disconnection of a pump to pneumatic massage elements 1346a-c by a women using one hand under her shirt without removing retainer 1347. Connector is optionally located in an unobtrusive location, for example below and/or between the breasts. Tubing and/or mechanical elements of the massage and/or collection system are optionally covered when not in use.

In some embodiments, a set of annular massaging elements is installed in each cup of the brassier (for example sewn between an inner and outer layer of the brassier). For example, each cup of retainer 1347 includes three annular pneumatic messaging elements 1346a-c. An extended underwire 1385 may circle under each cup of the brassier. The underwire 1385 may help hold the back end of the brassier and/or the messaging elements to the posterior side of the breast. Underwire 1385 optionally, crosses between the breasts, for example to hold the massaging elements to a posterior region in between the breasts.

In some embodiments, an opening 1349 is supplied to expose a nipple and/or an areola of the breast. For example, at the beginning of a milk expression session, a milk collecting flange 834 may be inserted through opening 1349. Optionally, flange 834 may be inserted without removing the brassier. In some embodiments the flange may be single use and/or disposable and/or multiuse. A user of the milk extraction system may insert the large end of the flange 834 through opening 1349. For example, flange 834 can be positioned over a nipple for milk collection and/or removed at the end of milk extraction while wearing and/or without removing retainer 1347. For example, as illustrated in FIG. 14A, opening 1349 may include offset vertical slits in the inner and/or outer layers of the brassier. Alternatively or additionally, slits may be at different angles, for example one slit may be vertical and the other horizontal. Retainer 1347 may optionally hold the flange over the nipple. For example, while the extraction system is in use, the slits may close over the flange pushing it against the breast and/or nipple (for example as illustrated in FIG. 13). Optionally, opening 1349 may include a fastener (for example, a button, a clasp, a zipper, a snap, a hook and loop fastener [for example Velcro™] and/or an adhesive). Optionally, opening 1349 may include a tie and/or an elastic closure. Optionally, opening 1349 may be adapted for a nursing pad. For example the size of opening 1349 and or the pad may be adapted for insertion of the pad. For example, the pad and/or retainer 1347 may include a fastener and/or a pocket to hold the pad in place.

In some embodiments, retainer 1347 may include a tubing connector 1487a. For example, tubing connector 1487a may be configured for quick connection and disconnection to a mating connector 1487b. Tubing connectors 1487a, b may optionally join input tubing 1465 to output tubing 1445a-c. For example, output tubing 1445a-c may join tubing connector 1487a to messaging elements (for example elements 1346a-c). For example, input tubing 1465 may join tubing connector 1487b to a pressure source, for example a pump. For example, tubing 1465 and/or tubing 1445 may have an internal diameter ranging between 1 to 3 cm and/or between 3 to 5 cm and/or between 5 to 7 cm and/or greater.

FIG. 14B contrasts some construction a convention brassier to a brassier for retaining a breast massager and/or milk collection apparatus according to some embodiments of the current invention. For example, lines 1451b show the upper edge of a conventional brassier (not designed for a breast massager) and 1451a show the higher upper edge of an exemplary massager retainer (for example retainer 1347). Optionally, the higher upper edge holds the top edge of the retainer 1347 and/or a massage element (for example the top of element 1356a) closer to the chest wall than the conventional brassier. In some embodiments, the under arm boundary 1453a of a massager retainer by be located further back than the under arm boundary 1453b of a conventional brassier not designed for massaging. For example, according to some embodiments of the current invention, the distance from the center of the cup of a retainer to the upper edge and/or the edge under the arm may range between 5 to 7 cm and/or 7 to 9 cm and/or between 9 to 11 cm. In some embodiments the point 1489a at which the upper edge of the retainer meets a shoulder strap may be higher up and further back than for some conventional brassieres (for example at 1489b). For example, in some embodiments, when a retainer is worn, the top of the breast cover may meet the shoulder strap behind a collarbone of a wearer. For example, in some embodiments, when a retainer is worn, the top of the breast retainer may cover at least part of the sternum and/or clavicle of a wearer. In some embodiments a retainer for a massager may include a strap adjustor 1477 and/or ring on the front of a shoulder strap, for example to better control the position of the top of the cup and/or breast cover and/or massaging element than on some brassieres not used as a massage retainer. In some embodiments, a massager retainer may include extra reinforcement 1469 under the arms of a user.

For bra not designed for massaging, an underwire 1485 may extend around the outside and bottom of the breast to push the breast inward in upward. For a massager retainer an underwire (for example underwire 1385) may optionally hold a massage element (for example element 1356a) at the root of the breast (for example preventing the massage element from being pushed up towards the nipple). In some embodiments of the current invention an underwire may also occupy space between the breasts, for example to hold the retainer near the chest wall at the center of the chest. For example, the underwire may hold massaging element in the space between the breast on the posterior region of the breast (for example, underwire 1385 as illustrated for example in FIGS. 13 and 14A). For example in some embodiments the underwire may reach the Xiphoid process of a wearer and/or the 7^{th} Costal notch of the wearer and/or the 6^{th} Costal notch of the wearer and/or the 5^{th} Costal notch of the wearer and/or the 4^{th} Costal notch of the wearer and/or the 3^{rd} Costal notch of the wearer and/or the 2^{nd} Costal notch of the wearer and/or the manubrium of the wearer.

### 12 Tubing Connector

FIG. 15 illustrates an external view of tubing connectors 1487a and 1487b according to an embodiment of the current invention. Connectors 1487a and 1487b may facilitate connection between massage elements and a power source, for example a source of pressurized air. For example, connector 1487a may be part of a massager retainer (for example retainer 1347). A mating connecter 1487b may be attached to a power source. For example, input tube receivers 1565a-c, a′-c′ of connector 1487b may be connected by tubes 1465 to a pressure source. Output tubes 1445a-c and/or 1445a′-c′ may be connected, for example, to pneumatic massaging elements for Example 1386a-c and/or their counterparts on a right breast. Optionally, connectors 1487a, b are designed to facilitate connecting under a shirt with and/or with one hand. For example, one or more of the couplers 1559 may be longer than the rest to make it easy to start aligning connectors 1487a,b. Clear areas 1557a, b (which have not tube connections) are optionally supplied for finger placement for squeezing together connectors 1487a, b between a thumb and finger. Connector 1487a optionally includes valves closing apertures 1559′ when they are not connected to mail couplers 1559. The valves may prevent water from entering tubes 1445a-c when retainer 1347 is washed. Alternatively or additionally a cover maybe supplied to cover apertures 1559′ when the retainer 1347 is washed.

FIG. 15B illustrates a cutaway view of tubing connector 1487a according to an embodiment of the current invention. In some embodiments, a tubing connector may include a distributer, directing flow and/or pressure to multiple outlets. For example, connector 1487a distributes pressurized air between six massage elements (for example elements 1346a-c on the left breast and a set of three similar elements on the right breast). Optionally, in connector 1467a five input apertures 1559′ exit to the massage elements in six exit tubes 1445a-c and 1445a′-c′. For example the center input tube receiver 1565c and/or aperture 1559′ may split between two exit tubes 1445c, c′. Alternatively or additionally connector 1487a may include male couplers 1559 and/or connector 1587b may include mating apertures. Optionally, the air supply to connector 1487b is controlled to achieve a desired sequence of messaging. For example air may first be introduced in to tube receivers 1565a, a′ filling element 1346a and/or a respective element on the right breast. Optionally air may be subsequently be introduced to tube receivers 1565b, b′ filling element 1346b and/or a respective element on the right breast. Optionally air may subsequently be introduced to tube receivers 1565c filling element 1346c and/or a respective element on the right breast. This may produce a wavelike massage pushing milk toward a nipple. In some embodiments after all elements 1346a-c are inflated they are all deflated together. For example the first massage element 1346a may be inflated over a period ranging between 0.5 and 2 seconds and then second massage element 1346b may be inflated over a period ranging between 0.5 and 2 seconds and then third massage element 1346c may be inflated over a period ranging between 0.5 and 2 seconds. Optionally the entire set of elements (1346a-c) will be kept inflated over a period ranging between 0.5 and 4 seconds. Optionally the entire set of massage elements will be deflated together over a period ranging between 0.5 and 4 seconds. Alternatively or additionally elements 1346a-c are deflated serially, for example in the same order that they are inflated. For example, after element 1346b is inflated element 1346a may be deflated and/or after element 1346c is inflated elements 1346b, c are deflated. Alternatively or additionally after all element 1346a-c are inflated, element 1346a may be deflated, for example over a time period ranging between 0.5 to 2 seconds, and/or then element 1346b may be deflated, for example, over a time period ranging between 0.5 to 2 seconds and/or then element 1346c may be deflated, for example over a time period ranging between 0.5 to 2 seconds.

### 13 Regulator

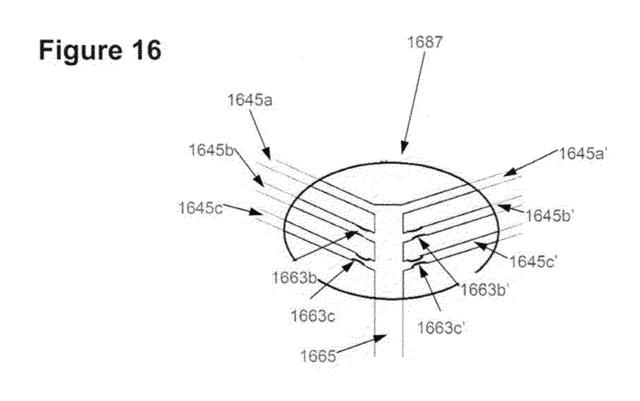

FIG. 16 illustrates a view of connections of a regulator included in a tubing connector for regulating time dependent pressure distribution according to an embodiment of the current invention.

In some embodiments, a tubing connector 1687 distributes pressure and/or flow from a single inlet to multiple outlets. The distribution of pressure may be time dependent. For example tubing connector 1687 has a single input tube 1665 and six output tubes 1645a-c and 1645a′-c′. Some of the output tubes have optional flow restrictions 1663c, b, c′, b′. When pressured air is applied to input tube 1665, air flows quickly into tubes 1645a, a′. Restrictions 1163b, b′ slow air flow into tubes 1645b, b′. Restrictions 1163c, c′ slow air flow into tubes 1645c, c′. For example, tubes 1645a, a′ may be connected to massage element 1346a and its counterpart on the right breast. For example, tubes 1645b, b′ may be connected to massage element 1346b and its counterpart on the right breast. For example, tubes 1645c, c′ may be connected to massage element 1346c and its counterpart on the right breast. For example, when air is pumped into input tube 1665 fast air flow in tubes 1645a,a′ may activate the corresponding massage elements (for example by inflating element 1346a) relatively quickly. Medium rate flow in tubes 1645b, b′ may active massage element 1346b more slowly. Slow rate flow in tubes 1645c, c′ may activate massage element 1346c more slowly. When pressure is released, air may drain through tubes 1645a-c, a′-c′. For example, massage element 1346a may be deactivated (for example deflated) quickly and/or massage element 1346a may be deactivated more slowly and/of massage element 1346a may be deactivated even more slowly. Alternatively or additionally, there may be a release valve that deactivates some or all of the massage units together, for example when the final massage element is activated; a valve may open deactivating all of the massage units together. Alternatively or additionally, a tubing connector may include different diameter tubes. For example, flow rates to different massage elements may be restricted according to the tube diameter (for example a large diameter tube to a massage element that is to be inflated first and a smaller tube to a massage element that is to be inflated later.

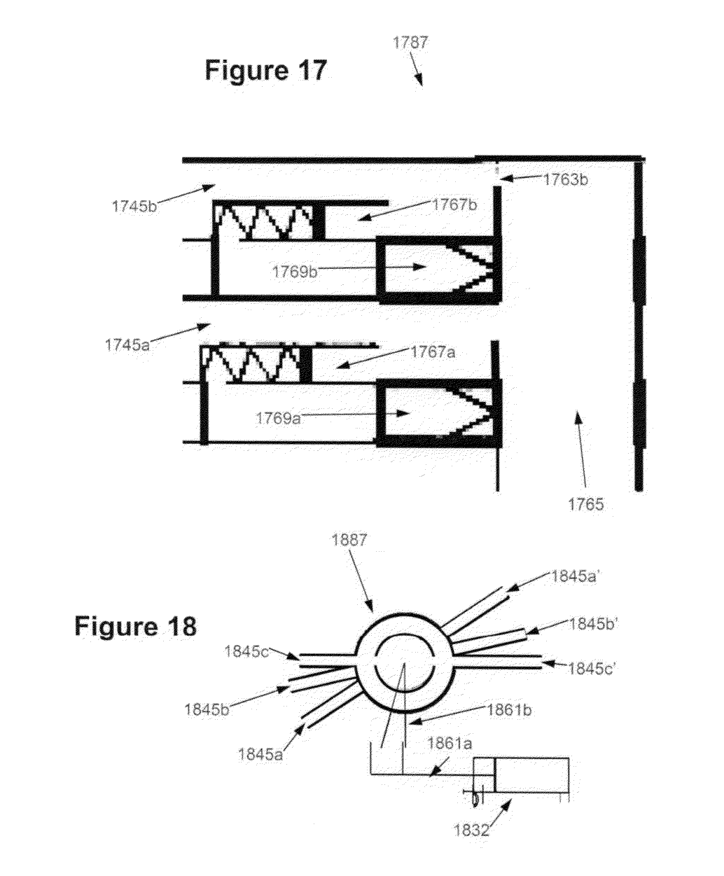

FIG. 17 illustrates a regulator 1787 that regulates time dependent flow for activating and/or deactivating massage elements according to some embodiments of the current invention. For example, a constant air pressure source may be supplied in an input tube 1765. Flow quickly enters an open outflow tube 1745a activating (for example by inflating) a first massage element (for example element 1346a). Pressure builds up until it reaches a predetermined value that causes a pressure release valve 1767a to open. When pressure release valve 1767a opens, pressure is optionally released for example through an outflow passage 1769a. Releasing pressure may deactivate (for example by deflating) an associated massage element. A second outflow tube 1745b is optionally connected to a second massage element (for example element 1346b). Outflow tube 1745b includes a flow restriction 1763b. Flow into tube 1745b may be slower than flow into tube 1745a. Optionally a respective second massage element is activated by air passing through restriction 1763b. In some embodiments the second massage element is activated more slowly than the first massage element. Pressure optionally builds up in outflow tube 1745b until a pressure release valve 1767b is released, releasing pressure for example through an outflow passage 1769b and/or deactivating the second massage element. Alternatively or additionally, a single pressure release valve may release pressure from multiple massage elements simultaneously. In some embodiments, a pressure release valve may be connected to a release pathway to release pressure directly to a pressure sink, for example a through tube to an outside atmosphere.

FIG. 18 illustrates an embodiment of a pump 1832 and a regulator distributer 1887 according to some embodiments of the current invention.

In some embodiments, a piston pump 1832 and distributer 1887 may be driven by one or more actuators. For example, in the embodiment a FIG. 18, a pump driving actuator 1861a is mechanically linked to an actuator 1861b controlling a rotary pressure distributor 1887. As pump 1832 progresses in its cycle, rotary distributor 1887 is rotated by actuator 1861b. Rotation of distributor 1887 optionally connects and/or disconnects various pairs of tubes 1845a-c, a′-c′ to the pressurized air stream by distributor 1887. Tubes 1845a-c, a′-c′ are optionally connected to various respective massaging elements which are activated or deactivated according to the applied pressure.

In some embodiments, massage elements may be individually controlled by a controller. For example, in the embodiment of FIGS. 4A and/or 15A,B a pressurization of individual massage elements may be controlled by a positive pressure pump and computer controlled valves individually controlling air flow in multiple tubes (for example tubes 445 and/or 1445a-c,a′-c′). In some embodiments, for example the embodiment of FIGS. 16 and/or 17 and/or 18, the pressure source may include a simple pump that pumps air and/or releases the air (for example to a single tube 1665 and/or tube 1765 and/or at a single rate and/or at a single pressure and/or produces fixed oscillations). In some embodiments, a tubing connector and/or a flow distributer and/or a flow regulator may be disposable. For example, a disposable flow distributer may be sold as a kit with other disposable parts of a milk collection system, for example a diaphragm and/or a collection bag and/or a collection flange.

It is expected that during the life of a patent maturing from this application many relevant technologies will be developed and the scope of the terms used are intended to include all such new technologies a priori.

As used herein the term “about” refers to ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

### EXAMPLES

Reference is now made to the following examples, which together with the above descriptions illustrate some embodiments of the invention in a non limiting fashion.

FIG. 19 illustrates an experimental apparatus for extracting breast milk in accordance with an embodiment of the present invention. In the experiment an optional retainer 1947 in the form of a brassier held pneumatic massage elements 1946a-c to a breast. A collection flange 834 was inserted through an opening in retainer 1947 and held over the breast.

In some experimental embodiments, message element 1946c was retained behind the flange 834 between the opening of flange 834 and 2.5 cm beyond the opening of flange 834; message element 1946b was retained behind the element 1946c between 2.5 cm beyond the opening of flange 834 to 5 cm beyond the opening of flange 834; message element 1946a was retained behind the element 1946b between 5 cm beyond the opening of flange 834 to 7.5 cm beyond the opening of flange 834. Pressure was directed from a pressure source through a connector 1987 and tubing 1945 to the individual massage elements 1946a-c.

The experimental positive pressure cycle directed to massage elements 1946a-c was according to an embodiment of the present invention. For example oscillating cycle started for 1 min with maximum pressure of about 60-80 mmHg and the maximum pressure was increased to 110-140 mmHg for the rest of the session. Air was pumped to each massage element 1946a-c at a constant rate, filling each message element until the desired maximum pressure value was reached. Inflating each massage element 1946a-c to the maximum pressure took approximately 1 sec. The message elements 1946a, 1946b and 1946c were inflated serially. After inflating all three elements 1946a-c all three elements 1946a-c where held inflated for 1-2 sec and deflated for 1-2 sec. each cycle took about 5-7 sec. filling volume varied according to breast.

In the experiment milk was extracted according to an embodiment of the present invention. In some cases, an oscillating vacuum was applied at a rate of 1 cycle per second. Optionally, in some cases, the vacuum cycle was synchronized with the massage cycle (every 5-7 sec). Optionally, the maximum vacuum of the cycle was kept at approximately 80 mmHg for a first period as long as the measured filling volume of the massage element increased over time. The filling volume was measured by measuring the time it takes the balloons to inflate since flow rate was approximately constant. In some embodiments, when the average time to fill balloons did not increase over 10 massage cycles (approximately 50-70 sec.) the first period was ended and extraction was stopped. In some embodiments, when the average time to fill balloons did not increase over 10 massage cycles (approximately 50-70 sec.) the first period was ended and a second extraction cycle was started. Optionally, the maximum vacuum was increased to 110 mmHg in the second period. Optionally, in the second period when the average time to fill balloons did not increase over 10 massage cycles (approximately 50-70 sec.) the second period was ended and extraction was stopped.

In some embodiments of the experiment the first period lasted from 6 to 10 minutes. Generally, the second period lasted from 2 to 5 minutes. For example the single period sessions lasted from 6 to 10 minutes and two period sessions lasted from 8 to 15 minutes. In most cases the subjects reported feeling that their breast had been sufficiently emptied.

FIG. 20 illustrates maximum vacuum 2092a over time and inflation time 2092b over time for an exemplary experimental example of a breast massager and breast milk extractor in accordance with an embodiment of the present invention. For example, in a first time period 2094 maximum vacuum 2092a was held at approximately 80 mmHg while the inflation time 2092b rose over a period of approximately 8 minutes to 1.2 seconds and stopped increasing. Optionally, when the inflation time leveled, a second period 2094b began. For example, in the second period, maximum vacuum 2092a was increased to approximately 120 mmHg. Increasing vacuum 2092a was associated with more milk extraction and further increase in inflation time 2092b for another 4 approximately 4 minutes. After 4 minutes (a total extraction time of 12 minutes), inflation time 2092b again leveled off and the extraction session was ended.

In a control group, milking sessions were conducted for twenty minutes to thirty minutes using the vacuum breast pump without massaging. In the control group the maximum vacuum in the extraction cycle varied between 180 to 250 mmHg.

In the experiment, twenty to thirty percent more milk was recovered in experimental sessions using massaging in accordance with some embodiments of the present invention than in control sessions. In the experiment, the milk extraction session was finished (the breast was emptied and more milk was recovered) in 30%-80% less time in experimental sessions using massaging in accordance with some embodiments of the present invention than in control sessions. In the experiment, milk extraction session used 30%-80% lower vacuum in experimental sessions using massaging in accordance with some embodiments of the present invention than in control sessions. In the experiment, women reported a more pleasant and comfortable experience in experimental sessions using massaging in accordance with some embodiments of the present invention than in control sessions. FIG. 20 illustrates the filling time and maximum vacuum over time of a typical two period experimental session in accordance with some embodiments of the present invention.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of extracting milk from a breast of a woman comprising:
securing a massager to the breast;
massaging a posterior region of the breast with said massager, said massaging continuing least intermittently over a massage time period; and
extracting milk from the breast during said massage time period.

2. The method of claim 1, further comprising:
positioning a flange over a nipple of said breast subsequent to said securing and wherein said extracting is through said flange.

3. The method of claim 2, wherein said positioning includes inserting said breast into said flange through an opening of said flange and wherein said massaging said posterior region includes a region posterior to said opening and at least 2 cm distant from said opening.

4. The method of claim 2, wherein said extracting includes applying an oscillating vacuum reaching a maximum magnitude of less than 100 mmHg over a first extracting period of at least four minutes.

5. The method of claim 2, wherein said extracting includes applying an oscillating vacuum over a first extracting period of at least four minutes and wherein during a maximum vacuum portion of said first extracting period a nipple of said breast is drawn into a nipple channel of said flange to a distance of less than 2 cm.

6. The method of claim 1, wherein said posterior region includes a location less than 3 cm from a chest wall of said woman.

7. The method of claim 1, wherein said posterior region includes three points defining a plane posterior to 50% of the volume of the breast.

8. The method of claim 1, further including:
detecting a volume of said breast; and
adjusting a parameter of said massaging in response to said detecting.

9. The method of claim 1, further including:
detecting a volume of said breast; and
adjusting a parameter of said extracting in response to said detecting.

10. A system for extracting milk from a woman comprising:
a breast massager;
a retainer securing said breast massager to a posterior region of a breast; and
an opening in said breast massager and retainer allowing access to a nipple of said breast.

11. The system of claim 10, wherein said opening is less than 6 cm wide.

12. The system of claim 10, further including:
a flange fitting through said opening and over said nipple, and
a collection unit collecting the milk from said flange into a collection vessel.

13. The system of claim 12, further including:
a source of a vacuum and wherein said flange includes a nipple channel and wherein said source is connected to said flange to draw said nipple into said nipple channel and wherein a length of said nipple channel is less than 2 cm.

14. The system of claim 12, wherein said retainer secures said massager to said posterior region of the breast, posterior to said flange and at a distance of at least 2 cm from said flange.

15. The system of claim 12 further comprising:
a controller programmed to direct massaging of said posterior region of said breast by said breast massager and simultaneously to direct said collecting the milk by said collection unit.

16. The system of claim 10, wherein said posterior region includes a location less than 3 cm from a chest wall of said woman.

17. The system of claim 10, wherein said posterior region includes three points defining a plane posterior to 50% of the volume of the breast.

18. The system of claim 10, wherein said massager includes one or more inflatable massage element.

19. The system of claim 18, wherein said retainer includes a channel and wherein upon inflation said massage elements fill said channel.

20. The system of claim 10, further comprising:
a breast volume sensor; and
a controller is configured to detect a change in volume of said breast from an output of said breast volume sensor.

21. The system of claim 20, further including:
a flange fitting through said opening and over said nipple, and
a collection unit collecting the milk from said flange into a collection vessel wherein said controller is configured to change a parameter of said collecting in response to said change in volume.

22. The system of claim 21, wherein said changing a parameter includes stopping said collecting.

23. The system of claim 20, wherein said controller is configured to change a parameter of said massaging in response to said change in volume.

24. The system of claim 20, wherein said massager includes an inflatable element and said sensor includes a pressure sensor for sensing a pressure of inflation of said inflatable element.

25. The milk extraction system of claim 20, further including:
an alerter controlled by said controller for notifying a user when a rate said change of said volume is below a threshold.
